(19) United States
(12) Patent Application Publication
(10) Pub. No.: US 2026/0108469 A1
(43) Pub. Date: Apr. 23, 2026

(54) COMPOSITIONS COMPRISING WATER INSOLUBLE DRUGS AND METHODS OF PRODUCING AND USING SAME

(71) Applicant: Nano Ghost Ltd, Netanya (IL)

(72) Inventors: Irina GOTLIV, Rehovot (IL); Haim TSUBERY, Elad (IL); Orit AMSALEM-BERGELSON, Rehovot (IL); Oded PINKAS, Petach-Tikva (IL); Yonatan MALCA, Tel Aviv (IL); Osnat BOHANA-KASHTAN, Tel-Mond (IL)

(73) Assignee: Nano Ghost Ltd, Netanya (IL)

(21) Appl. No.: 19/428,191

(22) Filed: Dec. 21, 2025

Related U.S. Application Data
(63) Continuation of application No. PCT/IL2023/050645, filed on Jun. 21, 2023.

(51) Int. Cl. A61K 9/50; A61K 47/64

(52) U.S. Cl. CPC A61K 9/5068; A61K 9/5089; A61K 47/64

(57) ABSTRACT

A composition is provided. The composition comprises a plurality of spherical particles composed of a whole cell membrane fraction, wherein the spherical particles exhibit native membrane symmetry and expression of native markers and wherein the plurality of spherical particles encapsulate a conjugate comprising a water insoluble drug linked to a cell penetrating peptide (CPP). Also provided are methods of producing and using the composition.

### RELATED APPLICATION

This application is a Continuation of PCT Patent Application No. PCT/IL2023/050645 having International filing date of Jun. 21, 2023. The contents of the above application are all incorporated by reference as if fully set forth herein in their entirety.

### SEQUENCE LISTING STATEMENT

The XML file, entitled 106135Sequence Listing.xml, created on Dec. 21, 2025, comprising 153,139 bytes, submitted concurrently with the filing of this application is incorporated herein by reference

### FIELD AND BACKGROUND OF THE INVENTION

The present invention, in some embodiments thereof, relates to compositions comprising water insoluble drugs and methods of producing and using same.

Targeted cancer therapy is the ultimate goal of the pharmaceutical industry. However, most platforms have relatively short circulation times and highly complicated production methods that hinder their clinical applications.

WO2011/024172 discloses a targeted delivery platform, based on nanoghosts (NGs) that are generated from the whole cell membrane of mesenchymal stem cells (MSCs). NGs derived from MSCs comprise typical MSC markers which ensure their targeting to cancer cells. In contrast to exosomes or other extracellular vesicles that are shed or bud from cells and their size is small (30-150 nm), MSC-NGs are manufactured in a reproducible controlled process by isolating intact MSC cell membranes (ghost cells), and homogenizing them into nanosized vesicles (nanoghosts) while entrapping a therapeutic of choice. This approach, has become a platform for active cancer-targeted drug delivery as it is biocompatible, characterized by long duration time within target site, and selectivity.

WO2022/264129 discloses an improved process for NGs production as well as methods of solubilizing water insoluble drugs, while avoiding leakage from loaded spherical particles.

### RELATED ART

Boone, C. W., Ford, L. E., Bond, H. E., Stuart, D. C. & Lorenz, D. Isolation of plasma membrane fragments from HeLa cells. J Cell Biol 41, 378-392 (1969).

Westerman and Jensen Methods Enzymol. 2003; 373:118-27.

Busatto, et al. Tangential flow filtration for highly efficient concentration of extracellular vesicles from large volumes of fluid. Cells 12:273 (2018)

Toledano-Furman et al. Reconstructed Stem Cell Nanoghosts: A Natural Tumor Targeting Platform. Nano Lett. 13:3248 (2013).

Kaneti et al. Nanoghosts as a Novel Natural Nonviral Gene Delivery Platform Safely Targeting Multiple Cancers. Nano Lett. 16:1574 (2016).

Timaner et al. Therapy-educated mesenchymal stem cells enrich for tumor-initiating cells. Cancer Res. 78:1253 (2018).

Oieni et al. Nano-ghosts: Novel biomimetic nano-vesicles for the delivery of antisense oligonucleotides. J Controlled Release 333:28 (2021).

Sepheri et al. Human serum albumin conjugates of 7-ethyl-10-hydroxycamptothecin (SN38) for cancer treatment. Biomed Res Int ID 963507 (2014).

Meyer-Losic et al. DTS-108, A Novel Peptidic Prodrug of SN38: In vivo Efficacy and Toxicokinetic Studies. Clin Cancer Res 14:2145 (2008).

Coriat et al. Pharmacokinetics and safety of DTS-108, a human oligopeptide bound to SN-38 with an esterase-sensitive cross-linker in patients with advanced malignancies: a Phase I study. Int J Nanomedicine 11:6207 (2016).

### SUMMARY OF THE INVENTION

According to an aspect of some embodiments of the present invention there is provided a composition comprising a plurality of spherical particles composed of a whole cell membrane fraction, wherein the spherical particles exhibit native membrane symmetry and expression of native markers and wherein the plurality of spherical particles encapsulate a conjugate comprising a water insoluble drug linked to a cell penetrating peptide (CPP).

According to some embodiments of the invention, the whole cell membrane faction is of mesenchymal stem cells (MSCs).

According to some embodiments of the invention, the water insoluble drug is selected from the group consisting of Camptothecin analogs (e.g. deruxtecan, topotecan, irinotecan/SN-38, and belotecan), Auristatin derivatives (e.g. MMAE: monomethyl auristatin E), Maytansinoids derivatives (e.g. M1/Emtansine/Mertansine), Anthracyclines (e.g. doxorubicin, daunorubicin, epirubicin and idarubicin), Platins (e.g. Cisplatin, Oxaliplatin, Carboplatin, Nedaplatin), Taxanes (Paclitaxel/Taxol/Docetaxel/Taxotere), Gemcitabine, Calicheamicin, Camptothecin and water-insoluble PARP inhibitors (e.g. Lynparza/Olaparib, Rubraca/Rucaparib, Zejula/Niraparib).

According to some embodiments of the invention, the water insoluble drug is camptothecin or an analog or derivative thereof.

According to some embodiments of the invention, the water insoluble drug is SN38.

According to some embodiments of the invention, the water insoluble drug is SN38 and the CPP is DPV1047.

According to some embodiments of the invention, the CPP is attached to the water insoluble drug via a linking group.

According to some embodiments of the invention, the linking group is 4-{4-[(N-maleimydomethyl)cyclohexanecarboxamido]methyl}cyclohexane-1-carboxylic acid linker (BCH).

According to some embodiments of the invention, the linking group is bifunctional.

According to some embodiments of the invention, the linking group is cleavable.

According to some embodiments of the invention, the linking group is non-cleavable.

According to some embodiments of the invention, the CPP is attached directly to the water insoluble drug.

According to some embodiments of the invention, the CPP is 7-25 amino acids in length.

According to some embodiments of the invention, the CPP comprises basic amino acids.

According to some embodiments of the invention, the CPP is of a human protein or a homolog or ortholog thereof.

According to some embodiments of the invention, the CPP is derived from a glucose amine glycan binding protein.

According to some embodiments of the invention, the CPP is derived from a heparin binding protein.

According to some embodiments of the invention, the CPP is selected from the group consisting of SEQ ID NO: 107-116.

According to some embodiments of the invention, the CPP is selected from the group consisting of DPV1047, N50, Short Penetratin, SV-40 (NLS), SynB3, Tat47-57, Tat49-57. DPV51 and CLIP6.

According to some embodiments of the invention, the CPP comprises at least one synthetic amino acid.

According to some embodiments of the invention, a backbone of the CPP comprises at least one modification.

According to an aspect of some embodiments of the present invention there is provided a method of producing a composition comprising a water insoluble drug, the method comprising:
(a) subjecting cells to a hypotonic treatment so as to obtain swollen intact cells;
(b) subjecting the swollen intact cells to flow shearing to obtain ruptured cells while avoiding nuclei lysis;
(c) filtering the ruptured cells to obtain a cell preparation devoid of nuclei;
(d) subjecting the cell preparation devoid of nuclei to purification by anion exchange chromatograpy, affinity chromatograpy, and/or filtration by size, to obtain ghosts, wherein the ghosts can be subjected to a further step of downsizing the ghosts using a high shear homogenizer or microfluidizer or extruder to obtain spherical particles of 35-400 nm;
(e) purifying the ghosts or spherical particles;
(f) incubating the spherical particles with a conjugate comprising a water insoluble drug linked to a cell penetrating peptide (CPP) under conditions which allow encapsulation of the conjugate in the particles; and
(g) removing free conjugate not undergoing the encapsulation.

According to some embodiments of the invention, a ratio of the spherical particles lipid and the conjugate in the incubating is about 10:1 to 1:10 mass ratio.

According to some embodiments of the invention, a ratio of the spherical particles and the conjugate in the incubating is about 2:1 to 1:2.

According to some embodiments of the invention, a ratio of the spherical particles and the conjugate in the incubating is about 1:1.

According to some embodiments of the invention, the conditions comprise 5-60 minutes of incubation.

According to some embodiments of the invention, the conditions comprise and 4-37° C.

According to some embodiments of the invention, the cells are mesenchymal stem cells (MSCs).

According to some embodiments of the invention, the MSCs having been cultured in suspension.

According to some embodiments of the invention, the cells are provided at an amount of at least 0.5×10⁹.

According to some embodiments of the invention, the MSCs exhibit a population doubling level (PDL) of 15-30.

According to some embodiments of the invention, the method further comprises culturing the cells prior to step (a).

According to some embodiments of the invention, the culturing is in a bioreactor.

According to some embodiments of the invention, the cells are a pure population of cells being ≥95% MSCs.

According to some embodiments of the invention, the hypotonic treatment is under dynamic conditions.

According to some embodiments of the invention, osmolarity of the hypotonic treatment is 5-100 mOsm/Kg.

According to some embodiments of the invention, the hypotonic treatment is effected for 10-60 minutes.

According to some embodiments of the invention, the hypotonic treatment results in cell swelling by at least 20% as determined by cell diameter.

According to some embodiments of the invention, the filtering the ruptured cells to obtain a cell preparation devoid of nuclei comprises a first filtration at a filter cut-off of 1.2-10 μm to remove nuclei, and optionally a second filtration at a filter cut-off of 0.45-0.85 μm to remove intracellular organelles.

According to some embodiments of the invention, the purification is performed by filtration, a size exclusion column, anion exchange and/or affinity chromatography.

According to some embodiments of the invention, the size separation is performed by Tangential Flow Filtration (TFF).

According to some embodiments of the invention, the method further comprises subjecting the ghosts to size separation following the downsizing.

According to some embodiments of the invention, the purifying comprises filtering the particles to obtain spherical particles at a filter cut-off of 0.05 μm.

According to an aspect of some embodiments of the present invention there is provided a composition comprising a plurality of spherical particles composed of a whole cell membrane fraction, wherein the spherical particles exhibit native membrane symmetry and expression of native markers and wherein the plurality of spherical particles encapsulate a conjugate comprising a water insoluble drug linked to a cell penetrating peptide (CPP) and wherein the composition is obtainable according to the method as described herein.

According to an aspect of some embodiments of the present invention there is provided a pharmaceutical composition comprising the composition as described herein and a pharmaceutically acceptable carrier or diluent.

According to an aspect of some embodiments of the present invention there is provided the pharmaceutical composition as described herein for use in treating cancer.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B shows expression of Cell Membrane MSC markers by nanoghosts (NGs). (A) Flow Cytometry dot plot analysis of NGs stained with antibodies against various surface MSC markers (including the MSC identity markers CD73, CD90, and CD105, that were defined by the international society of cell therapy). NGs do not express the pan hematopoietic marker CD45, while express various MSC surface integrins and adhesion molecules. (B) Bar graph of flow cytometry data presented in A.

FIGS. 2A-D show selective penetration of Alexa Fluor 488 labeled NGs to H460 human lung cancer cells. Flow Cytometry analysis of H460 lung cancer cells and PBMCs following incubation with Alexa Fluor 488 (AF488) labeled NGs. (A) Dot plot flow cytometry analysis of cells following uptake of NGs at various quantities (0.1-10 μg lipid/mL). (B-C) % Cells positive for AF488-NGs and the Mean Fluorescence Intensity (MFI) of AF488 positive cells, respectively. (D) Confocal imaging of SKOV-3 ovarian cancer cells following uptake of AF488-NGs.

FIG. 3 is an embodiment for the production steps of SN38-loaded NGs presented as a flow chart.

FIGS. 4A-B are chemical formulae of SN38 and an SN38-CPP Conjugate, respectively, according to some embodiments of the invention. (A) SN38 chemical structure, in the lactone active form. (B) SN38-CPP conjugate with ester linker.

FIGS. 5A-C show the in vitro efficacy of the SN38-CPP conjugate. (A) Release profile of SN38-CPP from loaded NGs, under sink conditions (dialysis against a high volume of TMS, at 37° C., with mixing). (B) Inhibition (IC50) of H460 NSCLC cells proliferation following treatment with NGs loaded with the SN38-CPP conjugate, as compared to free SN38, free SN38-CPP conjugate, free CPP (w/o SN38) and empty NGs (unloaded). Cells were incubated with the Drug/Control for up to 2 hrs or 6 hrs, Drug/Control were washed, and proliferation inhibition (IC50) was measured at 72 hrs from initial incubation. IC50 is summarized in the table of (c).

FIGS. 6A-C show the biodistribution of Cy7 labeled NGs in the HCT116 colorectal cancer (CRC) mouse model. Cy7 labeled NGs were IV injected daily for up to 5 days at a dose of 2 mg/kg to Balb/c Nude HCT116 CRC tumor bearing mice (subcutaneous tumor model). 2 hrs and 24 hrs post IV injection on Day 1, and 2 hrs post IV injection on Day 5 (after 5 daily injections), tumor and infiltrating organs were harvested, and imaged using the Vilber Newton 7.0 apparatus.

FIGS. 7A-B shows HCT116 CRC tumor growth inhibition by SN38-CPP loaded NGs. (A) Vehicle (TMS), unloaded NGs (2 mg/kg), SN38-CPP loaded NGs (2 mg NGs/kg) at two loading levels (1:0.5 and 1:0.75) yielding 0.2 and 0.5-0.7 mg SN38-CPP/kg, respectively, and free SN38-CPP at 0.7 mg/kg (the highest correlative amount given via loaded NGs), were injected IV, twice daily (BID), every other day (QOD), to Balb/c Nude HCT116 CRC tumor bearing mice (subcutaneous tumor model). SN38-CPP at 80 mg/kg was given 3 times a week and used as positive control. Tumor volume was measured by digital Caliper. (B) Mice body weight, normalized to pre-dose weight.

FIG. 8 is a schematic illustration of several suggested SN38 conjugates.

FIG. 9 is a schematic illustration of suggested conjugates.

### DESCRIPTION OF SPECIFIC EMBODIMENTS OF THE INVENTION

The present invention, in some embodiments thereof, relates to compositions comprising water insoluble drugs and methods of producing and using same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The majority of failures in new drug development has been attributed to poor water solubility of the drug. Issues associated with poor solubility can lead to low bioavailability resulting in suboptimal drug exposure.

The present inventors have now devised a new approach for increasing solubility and bioavailability of water insoluble drugs.

As is illustrated below and in the Examples section which follows, the present inventors have conjugated the water insoluble topoisomerase 1 inhibitor, irinotecan (or SN-38) to a cell penetrating peptide (CPP) and loaded it on un-cellular spherical particle (also termed “NanoGhosts” or NGs) and used the resultant composition in a series of in vitro and in vivo studies.

The present inventors have found that NGs encapsulating the conjugates exhibited about the same potency in eradicating a tumor in a mouse model for colorectal cancer as that of the free conjugate (unloaded SN38-CPP) when administered intravenously at an amount that is about 200 fold lower than the free conjugate.

Thus, according to an aspect of the invention there is provided a composition comprising a plurality of spherical particles composed of a whole cell membrane fraction, wherein the spherical particles exhibit native membrane symmetry and expression of native markers and wherein said plurality of spherical particles encapsulate a conjugate comprising a water insoluble drug linked to a cell penetrating peptide (CPP).

As used herein “spherical particles” relates to synthetic (as opposed to naturally occurring cell derived particles, e.g., exosomes) fully closed carrier molecules comprising a spherical lipid/protein bilayer membrane derived from cell membranes, in which an entrapped liquid volume is contained. Thus, these spherical particles of the present invention are also referred to herein, as cell derived liposomes (CDLs). The spherical particle can be freeze-dry lyophilized and re-dehydrated to obtain their initial structure.

The spherical particles include niosomes, transfersomes, emulsions, foams, micelles, liquid crystals, dispersions, lamellar layers and the like.

The liposomes may be unilamellar or multilamellar.

According to a specific embodiment of the invention, the liposomes are unilamellar, as determined by Cryo-TEM and shown also in Furman et al., Nano Lett. Reconstructed Stem Cell Nanoghosts: A Natural Tumor Targeting Platform” (2013), Vol. 13, p. 3248-3255, Kaneti et al., Nano Lett. “Nanoghosts as a Novel Natural Nonviral Gene Delivery Platform Safely Targeting Multiple Cancers” (2016), Vol. 16, p. 1574-1582 as well as in WO 2011/024172, each of which is incorporated by reference in its entirety.

According to a specific embodiment of the invention, the spherical particles exhibit native membrane symmetry and expression of native markers. As used herein “membrane symmetry” means the orientation of membrane components, which is exhibited by the cell plasma membrane in a non-ruptured membrane, under physiological conditions.

Liposomes of the present invention are composed of a whole cell membrane fraction.

According to some embodiments, the spherical particles are referred to as NanoGhosts (NGs).

The spherical particles of some embodiments of the present invention have an expected protein to lipid ratio higher than about 0.8 w/w, e.g., 0.9-5 w/w, 0.9-4.5 w/w, 0.9-5 w/w, 0.9-4 w/w, 0.9-3.5 w/w, 0.9-3 w/w, 0.9-2.9 w/w, 0.9-2.8 w/w, 0.9-2.7 w/w, 0.9-2.6 w/w, 0.9-2.5 w/w, 0.9-2.4 w/w, 0.9-2.3 w/w, 0.9-2.2 w/w, 0.9-2.1 w/w, 0.9-2.9 w/w, 0.9-2.0 w/w, 0.9-1.9 w/w, 0.9-1.8 w/w, 0.9-1.7 w/w, 0.9-1.6 w/w, 0.9-1.5 w/w, 0.9-1.4 w/w, 0.9-1.3 w/w, 0.9-1.2 w/w, 1-2.7 w/w, 1.1-2.7 w/w, 1.2-2.7 w/w, 1.3-2.7 w/w, 1.4-2.7 w/w, 1.5-2.7 w/w, 1.6-2.7 w/w, 1.7-2.7 w/w, 1.8-2.7 w/w, 1.9-2.7 w/w, 2.0-2.7 w/w, 2.1-2.7 w/w, 2.2-2.7 w/w, 2.3-2.7 w/w, 2.4-2.7 w/w, 2.5-2.7 w/w, e.g., about 2.5 w/w (for an unloaded spherical particles).

Protein and lipid assessments can be done using methods which are well known in the art, such as BCA, Bradford for protein and Stewart phospholipids and choline assessment assays for lipid.

According to a specific embodiment the protein content is determined using a BCA Protein assay or Smith assay (e.g., kits are commercially available such as from ThermoFisher) and lipid assays (e.g., kits are commercially available such as from Abcam and Sigma) and are typically carried out using commercial kits according to manufacturer's instructions.

According to some embodiments, the protein content of hMSCs spherical particles generated according to some embodiments of the invention is at least about 0.8 mg/10⁹ cells (as determined by a micro BCA protein assay) to about 81 mg/10⁹ cells. The lipid content is 1-30 mg per 10⁹ cells.

As used herein the phrase “cell membrane” or “cellular membrane” (which may be interchangeably used) refers to a biological membrane, essentially that which surrounds the cell.

The use of plasma membrane is of a specific advantage since it presents proteins, which are associated with cell-to-cell interactions as well as other recognition molecules, such as adhesion molecules, integrins, and receptors that bind soluble ligands and may elicit beneficial therapeutic properties such selective binding and immune modulation.

The cell membrane comprises both lipids and membrane-anchored proteins, and may be referred to herein as “whole cell membrane” to distinct from lipids alone or membrane proteins alone.

Examples of membrane proteins include, but are not limited to, integral proteins, transmembrane proteins, lipid anchored proteins and glycoproteins.

According to a specific embodiment, the particles comprise glycosaminoglycans (GAGs).

The present inventors have uncovered that a GAG-binding CPP is able to transfer its payload to NGs, despite the fact that GAGs (e.g., heparin) are mainly pericellular.

According to an embodiment of the invention, the whole cell membrane fraction also includes carbohydrates.

According to a specific embodiment the cell is a eukaryotic cell [e.g., mammalian (such as human), plant, insect cell].

According to an additional specific embodiment, the eukaryotic cell is a mammalian cell.

According to yet an additional embodiment the cell can be a primary cell (i.e., non-immortalized and at times not cultured) or a cell-line.

According to yet an additional embodiment the cell can be an embryonic cell.

According to yet an additional embodiment the cell can be a fetal, post-natal or adult cell.

According to a specific embodiment, the cell is a terminally differentiated cell.

Use of a primary cell may be advantageous for clinical use where non-cultured cells are used in autologous or non-autologous (syngeneic allogeneic or xenogeneic) settings.

According to a specific embodiment the eukaryotic cell is a stem cell.

As used herein, the phrase “stem cells” refers to cells, which are capable of remaining in at least somewhat undifferentiated state (e.g., progenitor, pluripotent or multipotent stem cells) or induced to undifferentiated (e.g., induced pluripotent stem cells (iPSCs)) for extended periods of time in culture until induced to differentiate into other cell types having a particular, specialized function (e.g., terminally differentiated cells). Preferably, the phrase “stem cells” encompasses embryonic stem cells (ESCs), induced pluripotent stem cells (iPS), adult stem cells, mesenchymal stem cells and hematopoietic stem cells.

According to a specific embodiment the stem cell is a mesenchymal stem cell.

Mesenchymal stem cells are the formative pluripotent blast cells. Mesenchymal stem cells (MSCs) give rise to one or more mesenchymal tissues (e.g., adipose, osseous/bone, cartilaginous, elastic and fibrous connective tissues, myoblasts, cardiac like cells) as well as to tissues other than those originating in the embryonic mesoderm (e.g., neural cells) depending upon various influences from bioactive factors such as cytokines. MSCs can be isolated from embryonic yolk sac, placenta, umbilical cord, fetal and adolescent skin, blood, bone marrow, adipose and other tissues, although their abundance in the bone marrow far exceeds their abundance in other tissues. MSCs have been shown to have immunosuppressive functions in various settings, including autoimmune diseases and transplantation, rendering liposomes generated therefrom ultimate tools in inflammatory and autoimmune settings.

Methods of isolating, purifying and expanding mesenchymal stem cells (MSCs) are known in the arts and include, for example, those disclosed by Caplan and Haynesworth in U.S. Pat. No. 5,486,359 and Jones E. A. et al., 2002, Isolation and characterization of bone marrow multipotential mesenchymal progenitor cells, Arthritis Rheum. 46(12): 3349-60.

Mesenchymal stem cell cultures can be generated by diluting for example BM aspirates with equal volumes of Hank's balanced salt solution (HBSS; GIBCO Laboratories, Grand Island, NY, USA) or PBS that contains EDTA and layering the diluted cells over Ficoll (Ficoll-Paque; Pharmacia, Piscataway, NJ, USA). Following 30 minutes of centrifugation at 2,500×g, the mononuclear cell layer is removed from the interface and suspended in HBSS or PBS. Cells are then centrifuged at 1,500×g for 15 minutes and resuspended in xeno free Rooster Nourish-MSC-XF Expansion Medium. Resuspended cells are plated in about 25 ml of medium in a 10 cm culture dish (Corning Glass Works, Corning, NY) or cellbind flasks and incubated at 37° C. with 5% humidified CO₂. Following 24-48 hours in culture, nonadherent cells are discarded, and the adherent cells are thoroughly washed twice with phosphate buffered saline (PBS). The medium is replaced with a fresh complete medium every 3 or 4 days for up to 14 days. Adherent cells are then harvested with 0.25% trypsin and 1 mM EDTA (Trypsin/EDTA, GIBCO) or TrypLE Select for 5 min at 37° C., replated in a 6-cm plate or T225 cell bind flasks and cultured for up to 5 days. Cells are then trypsinized and counted using a cell counting device such as for example, a hemocytometer (Hausser Scientific, Horsham, PA) or NC-200 cell counter. Cultured cells are recovered by centrifugation and resuspended with 5% DMSO and 30% FCS or with CryoStor CS5 or CS10 freezing medium at a concentration of 1 to 2×10⁶ cells per ml. Aliquots of about 1 ml each are slowly frozen and stored in liquid nitrogen (LN2) or LN2 vapor phase.

To expand the mesenchymal stem cell fraction, frozen cells are thawed at 37° C., diluted with a complete medium and recovered by centrifugation to remove the DMSO. Cells are resuspended in a complete medium and plated at a concentration of about 2,000-5,000 cells/cm². Under these conditions, MSC cultures can grow for about 50 population doublings and be expanded for about 2000-fold [Colter DC., et al. Rapid expansion of recycling stem cells in cultures of plastic-adherent cells from human bone marrow. Proc Natl Acad Sci USA. 97:3213-3218, 2000]. An exemplary embodiment for MSCs expansion is provided in the Examples section.

MSC cultures utilized by the present invention preferably include three groups of cells, which are defined by their morphological features: small and agranular cells (referred to as RS-1, herein below), small and granular cells (referred to as RS-2, herein below) and large and moderately granular cells (referred to as mature MSCs, herein below). The presence and concentration of such cells in culture can be assayed by identifying a presence or absence of various cell surface markers, by using, for example, immunofluorescence, in situ hybridization, and activity assays.

When MSCs are cultured using methods which are well known in the art they exhibit negative staining for the hematopoietic stem cell markers CD34, CD19/CD79a, CD11B, and CD45. A small fraction of cells (less than 5%) are dimly positive for CD31 and/or CD38 markers. In addition, mature MSCs are dimly positive for the hematopoietic stem cell marker, CD117 (c-Kit), moderately positive for the osteogenic MSCs marker, Stro-1 [Simmons, P. J. & Torok-Storb, B. (1991). Blood 78, 5562] and positive for the thymocytes and peripheral T lymphocytes marker, CD90 (Thy-1). On the other hand, the RS-1 cells are negative for the CD117 and Strol markers and are dimly positive for the CD90 marker, and the RS-2 cells are negative for all of these markers.

According to a specific embodiment the MSCs are positive for the CD markers CD73, CD90, and CD105 and additionally the MSCs are negative for the CD markers CD34, CD45, CD11b, CD79alpha/CD19, and HLA-DR.

Other cells, which may be used as an effective source for whole cell membrane fraction include, but are not limited to, endothelial cells, hepatic cells, pancreatic cells, bone cells, chondrocytes, neuronal cells, immune cells (e.g., lymphocytes, e.g., T-lymphocytes), fibroblasts and the like.

The cells can be used native (i.e., not manipulated by genetic modification) or genetically modified to manipulate the membrane composition of the cell.

Such a genetic modification can ensure correct/enhance targeting or a therapeutic function (e.g. ectopic expression of an antibody such as anti PDL1). According to some embodiments, it does not refer to the loaded agent as further described hereinbelow but to an ingredient from which the spherical particles are formed. Nonetheless, such as an expression product may have a therapeutic value.

The advantage of genetic modification is in its increased efficiency. Essentially all (>95%) the spherical particles generated from genetically modified cells express the gene-of-interest. The gene-of-interest may be constitutively expressed on the cell source (by integration to the cells genome) or transiently expressed (episomal expression) such as to avoid hazardous implications of stable transfection agents (e.g., lentiviral, adenoviral and retroviral vectors).

Thus, the cells may be genetically modified to express an exogenous (i.e., heterologous) gene-of-interest (i.e., either not naturally expressed in the native membrane or in order to enhance the expression of endogenous proteins that are naturally expressed on the native cell's membrane but in lower levels).

According to specific embodiments, the gene-of-interest encodes a membrane protein. The gene-of-interest may be a native membrane protein or modified to have a membrane localization signal and other motifs needed for membrane anchorage e.g., a transmembrane domain.

Examples of membrane proteins which may be heterologously expressed include, but are not limited to, a targeting protein (e.g., antibodies, receptors, membrane anchored ligands, decoys), a protein which affects the chemistry of the membrane (e.g., structural proteins, charged proteins), a diagnostic protein (e.g., an enzyme as described in length below) or a therapeutic protein (as described in length in WO2022/264129, which is hereby incorporated by reference in its entirety).

A targeting moiety includes a targeting protein such as an antibody, a receptor ligand and a non-proteinecious molecule such as carbohydrates, which binds cell surface or extra-cellular matrix markers. For example, prostate-specific membrane antigen (PSMA) that is over-expressed on prostate cancer cells can be targeted by its ligand NAAG3 conjugated to a transmembranal motif (e.g, truncated LIME)+. This may be achieved, by genetically engineering the cells (of which the CDLs are derived from) to express the chimeric or natural form of NAAG. For example, the expression plasmid encoding LIME is constructed by PCR and subsequent insertion of the corresponding fragment into pcDNA3.1 (Invitrogen). The primers also have BamHI (5′ primer and 3′ primer) site extension to facilitate the subcloning. The PCR product is digested with BamHI and inserted into corresponding sites in pcDNA3.1 (+) (CLONTECH Laboratories, Inc.). For expression vector encoding LIME-acetylaspartylglutamate (NAAG), the open reading frame can be inserted into plasmid coding LIME such that the NAAG is conjugated through its N-terminus and maintains its C-terminus free to react with PSMA [i.e., LIME(C)-(N)NAAG-COOH]. Alternatively, expression plasmid encoding NAAG-LIME chimera can be constructed following the method described previously described for CD8-LIME chimera⁵. Fragments corresponding to NAAG and LIME transmembrane region were generated by PCR. Primers encoding the 3′ sequences of the NAAG and the 5′ sequences of the LIME fragment were designed to overlap, such that annealing of the two products yielded a hybrid template. From this template, the chimera is amplified using external primers containing XbaI sites. The NAAG-LIME chimera is inserted into pcDNA3.1 (+).

As used herein, the phrase “surface marker”, refers to any chemical structure, which is specifically displayed at uniquely high density, and/or displayed in a unique configuration by a cell surface or extracellular matrix of the target cell/tissue.

For example, the targeting moiety may be useful for targeting to tumor cells. For example, it is generally accepted that the intracellular environment of tumor cells is more alkaline compared to their immediate extracellular environment, which in turn is more acidic than the microenvironment found in the angiogenic blood vessels feeding the tumor. In addition, many previous studies have shown that the surface charges of tumor cells are more negative compared to benign normal cells and even less invasive tumor cells. Accordingly, it may be useful to express membrane-bound enzymes and/or proteins, which will render the liposomes with a positive charge only in the acidic intermediate extracellular environment of the tumor. For example, any membranal protein with a pI of about 7.2-7.4 that falls between the high alkaline pH of the angiogenic blood vessels (pH>7.4) and the low acidic pH of the tumor immediate extracellular environment (pH<7.2) can be used. Such proteins can be specifically identified by cross referencing the RCSB Protein Data Bank (PDB) for human plasma membrane proteins. The expected desirable pI (7.2-7.4) for those proteins can be calculated using the standard iterative algorithm that gives relatively precise results of pI calculations for raw protein sequences. The algorithm is used in the Compute pI/Mw tool at the ExPASy server. Such liposomes are expected to have negative or neutral charge in the alkaline microenvironment of the angiogenic tumor vessels and positive charge in the more acidic immediate extracellular environment of the tumor. Accordingly, this charge alteration will assist both liposomal extravasation, which is significantly enhanced for negative of neutral particles, and intra-tumor delivery which is more easily accomplished with positively charge particles.

Ample guidance regarding surface markers specifically over-expressed in diseases such as cancer, and antibodies specific for such surface markers is provided in the literature of the art (for example, refer to: A M Scott, C Renner. “Tumour Antigens Recognised by Antibodies.” In: Encyclopedia of Life Sciences, Nature Publishing Group, Macmillan, London, UK, www(dot)els(dot)net, 2001).

Diseases associated with a target cell/tissue specifically displaying a growth factor receptor/TAA surface marker which are amenable to treatment by the method of the present invention include, for example, some of the numerous diseases which specifically display growth factor receptors/TAAs, such as EGF receptor, platelet derived growth factor (PDGF) receptor, insulin like growth factor receptor, vascular endothelial growth factor (VEGF) receptor, fibroblast growth factor (FGF) receptor, transferrin receptor, and folic acid receptor.

In a specific embodiment, the ligand is an antibody or an antibody fragment, targeting antigens specific to a receptor on a target cell. Antibodies can be monoclonal antibodies, polyclonal antibodies or antibody fragments, which are target specific. In an embodiment, the antibodies attached to the liposomes are anti-CD19, anti-CD20, or anti-CD22, for specific binding to a B-cell epitope. These antibodies or antibody fragments are typically derived from hybridomas that show positive reactivity toward the affected B-cells. It is contemplated that other antibodies or antibody fragments targeting any other cell in the body can be similarly used. For example, anti-CD19 antibodies are used to target liposome containing an entrapped agent to malignant B-cells. The antibody recognizes a unique epitope, the CD19 surface antigen, on the B-cells. Alternatively or additionally, the antibody or targeting moiety can target a checkpoint molecule. Examples of checkpoint molecules which are stimulatory or inhibitory include, but are not limited to, PD-1, PD-L1, CTLA4, CD27, CD40, OX40, GITR, B7-CD28, ICOS and CD137.

Methods of expressing heterologous proteins in eukaryotic cells are well known in the art.

Thus, an exogenous polynucleotide sequence designed and constructed to express at least a functional portion of the gene-of-interest may be expressed in the cells from which membranes are later extracted. Accordingly, the exogenous polynucleotide sequence may be a DNA or RNA sequence of the gene-of-interest.

The phrase “functional portion” as used herein refers to part of the encoded protein (i.e., a polypeptide), which exhibits functional properties of the enzyme such as binding to a substrate. For example, the functional portion of an antibody may be the variable region conferring specificity and additional/or alternatively the constant region, i.e., Fc, which may activate complement and induce cell killing. For example, cells can be transfected with genes encoding one or more members from the GPCRs family (e.g., CCR5, CXCR4 etc.) that will render the liposomes targeted against abundant of cellular pathologies including auto-immune and viral 30 diseases (e.g., HIV/AIDS).

To express exogenous gene-of-interest in eukaryotic (e.g., mammalian) cells, a polynucleotide sequence encoding the gene-of-interest is preferably ligated into a nucleic acid construct suitable for eukaryotic cell expression. Such a nucleic acid construct includes a promoter sequence for directing transcription of the polynucleotide sequence in the cell in a constitutive or inducible manner.

Alternatively, cells, membranes, ghosts or the spherical particles derived therefrom, may be chemically treated such as to present a protein, a saccharide, a synthetic polymer, a peptide or any combination of same. Methods of modifying the membrane with a synthetic polymer are described hereinbelow. Such a chemical attachment may be effected at any stage from live cultured or suspended cells to produce the spherical particles.

For example, the spherical particles may be also chemically conjugated with folate that may further enhance their targeting and attachment to tumor cells, which are known to express higher levels of folate receptors compared to benign cells.

According to another example, it is also possible to permanently modulate the spherical particles to have a more positive surface charge by treating them with cations, salts or polycations (e.g., Polybrene®, polyethyleneimine and Poly-L-Lysine) rendering them more positive to better target the tumor angiogenic vasculature.

Non-native material can be also introduced to the surface of the spherical particles by fusion (e.g., PEG or detergent induced) with other liposomes (e.g., cell-derived or synthetic) that may be comprised of well characterized lipids, proteins and additives. Such a fusion, creating hybrid spherical particles, can be used to conjugate any moieties (e.g., targeting, therapeutic, diagnostic, stealth-rendering etc.) to the CDLs and to alter their surface properties. Synthetic polymers are typically used to prevent or reduce coagulation, increase dispersion, reduce interaction with blood components, evade non-specific uptake by the mononuclear phagocytic system and prolong the particle circulation time to a large extent thus, rendering the spherical particles with properties and features that are commonly referred to as stealth properties or long-circulating liposomes. Accordingly, the pH nano-environment at the particle surface may also be dependent upon the length of these molecules.

There are numerous polymers, which may be attached to lipids. Polymers typically used as lipid modifiers include, without being limited thereto: polyethylene glycol (PEG), polysialic acid, polylactic (also termed polylactide), polyglycolic acid (also termed polyglycolide), apolylactie-polyglycolic acid’ polyvinyl alcohol, polyvinylpyrrolidone, polymethoxazoline, polyethyloxazoline, polyllydroxyetlyloxazolille, solyhydroxypryloxazoline, polyaspartarllide, polyhydroxypropyl methacrylamide, polymethacrylamide, polydimethylacrylamide, polyvinylmethylether, polyhydroxyethyl acrylate, derivatized celluloses such as hydroxymethylcellulose or hydroxyethylcellulose.

The polymers may be employed as homopolymers or as block or random copolymers.

The most commonly used and commercially available lipids derivatized into lipopolymers are those based on phosphatidyl ethanolamine (PE), usually distearylphosphatidylethanolamine (DSPE).

A specific family of lipopolymers, which may be employed by the invention include PEG-DSPE (with different lengths of PEG chains) in which the PEG polymer is linked to the lipid via a carbamate linkage and Polyethyleneglycol distearoylglycerol. The PEG moiety headgroup preferably has a molecular weight from about 750 Da to about 20,000 Da. More preferably, the molecular weight is from about 750 Da to about 12,000 Da and most preferably between about 1,000 Da to about 5,000 Da. Two exemplary DSPE-PEG are those wherein PEG has a molecular weight of 2000 Da, and of 5000a designated herein DSPE-PEG (2000) (DSPE-PEG2k) and DSPE-PEG (5000) (DSPE-PEG5k).

Specific families of lipopolymers, which may be also employed by the invention, include C8 and C16 mPEG Ceramides (with different lengths of PEG chains) in which the PEG-Ceramides contain ester linkages between the PEG and ceramide moieties that allow the compound to be easily metabolized. The PEG moiety headgroup preferably has a molecular weight from about 750 Da to about 2,000 Da. More preferably, the molecular weight is about 2,000 Da.

Conventional post-insertion PEGylation of common liposomes requires heating or solublization in a detergent containing solution that might damage surface proteins and lead to encapsulate leakage. Therefore, spherical particles may be also PEGylated by the two following described methods or their combination. Primarily, PEGylated spherical particles will be prepared 20) by detergent-dialysis incorporation of PEGylated lipids into the ghost cell membrane (prior to spherical particles preparation). Following, direct PEGylation of the spherical particles may be performed with monomethoxy-PEG activated by succinimidyl succinate, which has been proven to increase the transfection efficiency and reduce serum mediated inactivation of PEGylated lentiviral particles, used as gene transduction vectors¹⁶.

Chemical binding of non-proteinaceous components (e.g., synthetic polymers, carbohydrates and the like) to the spherical particles surface may be employed. Thus, a non-proteinaceous moiety, may be covalently or non-covalently linked to, embedded or adsorbed onto the spherical particles using any linking or binding method and/or any suitable chemical linker known in the art. The exact type and chemical nature of such cross-linkers and cross linking methods is preferably adapted to the type of affinity group used and the nature of the liposome. Methods for binding or adsorbing or linking the enzyme and/or targeting moiety are also well known in the art.

For example, the enzyme and/or targeting moiety may be attached to a group at the interface via, but not limited to, polar groups such as amino, SH, hydroxyl, aldehyde, formyl, carboxyl, His-tag or other polypeptides. In addition, the enzyme and/or targeting moiety may be attached via, but not limited to, active groups such as succinimidyl succinate, cyanuric chloride, tosyl activated groups, imidazole groups, CNBr, NHS, Activated CH, ECH, EAH, Epoxy, Thiopropyl, Activated Thiol, etc. Moreover, the enzyme and/or targeting moiety may be attached via, but not limited to, hydrophobic bonds (Van Der Waals) or electrostatic interactions that may or may not include cross-linking agents (e.g., bivalent anions, poly-anions, poly-cations etc.).

As used herein, the terms “conjugate” or “conjugated” refer to a covalent, or non-covalent, ionic, or hydrophobic interaction whereby the moieties of a molecule are held together and preserved in proximity and in this case the CPP and the water insoluble drug.

According to a specific embodiment, the conjugate is a covalently bound conjugate.

As used herein “water insoluble drug” also known as a poorly water-soluble drug, refers to a pharmaceutical compound or substance that has limited or minimal ability to dissolve in water. Typically, when such a drug is added to water or an aqueous solution, it does not readily dissolve and instead forms particles or aggregates that may settle or remain suspended in the liquid. According to some embodiments, the water solubility is defined as below than 1 mg/ml. According to other embodiments, below than 100 μg/ml. According to other embodiments, below than 10 μg/ml.

Examples of such water-insoluble drugs include, but are not limited to:
Camptothecin analogs (e.g. deruxtecan, topotecan, irinotecan/SN-38, and belotecan), Auristatin derivatives (e.g. MMAE: monomethyl auristatin E), Maytansinoids derivatives (e.g. M1/Emtansine/Mertansine), Anthracyclines (e.g. doxorubicin, daunorubicin, epirubicin and idarubicin), Platins (e.g. Cisplatin, Oxaliplatin, Carboplatin, Nedaplatin), Taxanes (Paclitaxel/Taxol/Docetaxel/Taxotere), Gemcitabine, Calicheamicin, Camptothecin and water-insoluble PARP inhibitors (e.g. Lynparza/Olaparib, Rubraca/Rucaparib, Zejula/Niraparib).

According to a specific embodiment, the water-insoluble drug is a camptothecin analog.

According to a specific embodiment, the water-insoluble drug is SN-38, a topoisomerase 1 inhibitor that leads to generation of DNA breaks and induction of cell death in cells undergoing proliferation. It is a highly potent drug with high systemic toxicity and compromised bioavailability. SN-38 is FDA approved as the prodrug Irinotecan (CPT-11) for colorectal cancer, as liposomal Irinotecan formulation (Onivyde, Ipsen) for pancreatic cancer and as the antibody drug conjugate (ADC) Sacituzumab Govitecan-hziy (aTrop-2/Immunomedics IMMU-132) for triple negative breast, bladder and urinary tract cancers. SN-38 clinical application as a small molecule drug product was hindered by poor solubility, low plasmatic stability, and severe toxicity. SN-38 ADCs overcome some of these limitations but are limited to the tumors expressing their target antigen. In addition, it is estimated that due to biodistribution, uptake and loss of conjugation in circulation, only 1-2% of ADC payload will reach the intracellular target.

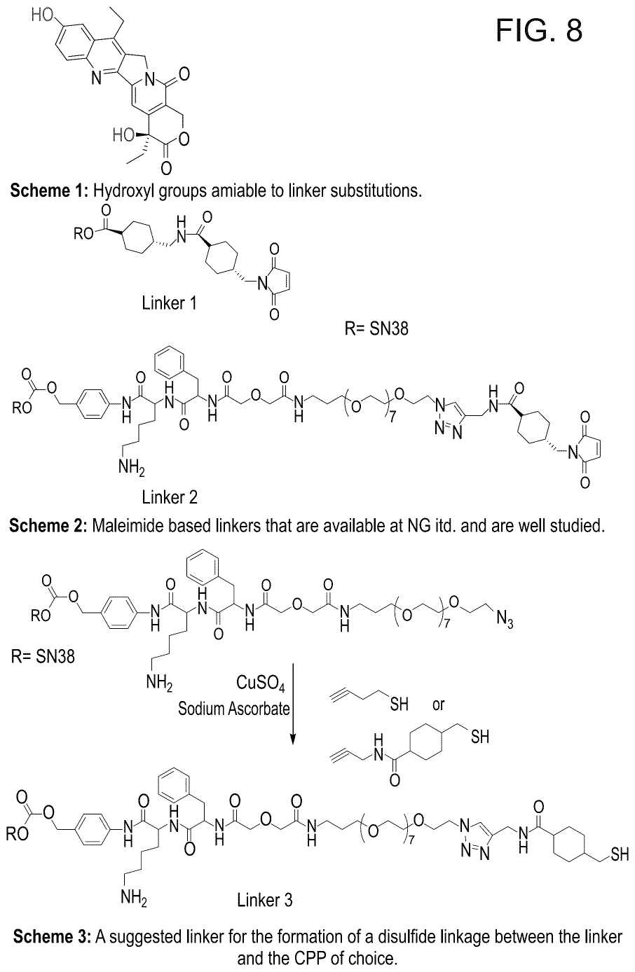

Embodiments of the invention rely on the conjugation of SN38 to CPP (also known in the art as well as on FIG. 8, for example) and loading it on NGs.

According to a specific embodiment, the conjugate is DTS-108, which comprises a CPP as DPV1047 in the following form: DPV1047-linker-SN38 (DPV-1047 is the peptide VKRGLKLRHVRPRVTRMDV (SEQ ID NO: 8); SN38 is 7-ethyl-10-hydroxycamptothecin); and the linker is 4-{4-[(N-maleimydomethyl)cyclohexanecarboxamido]methyl}cyclohexane-1-carboxylic acid linker (BCH), and the linker is attached to the peptide via an addition of a Cys residue at the N-terminal of the peptide.

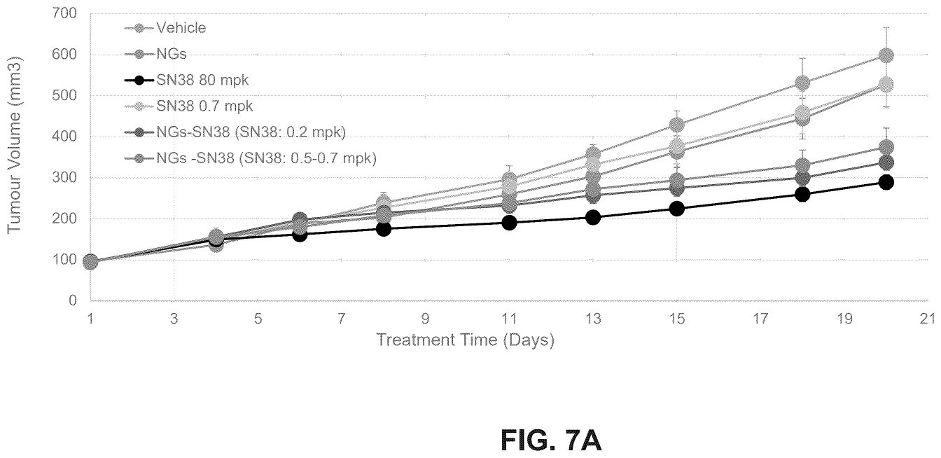

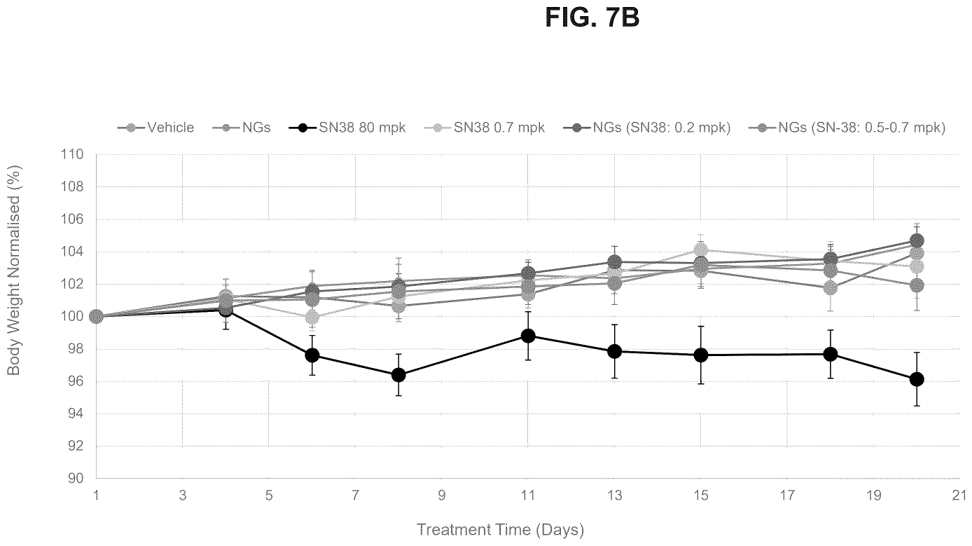

It is expected that this will broaden the use of SN-38, and possibly allow delivery of higher SN-38 amounts to cancer cells, as compared to ADCs, as supported by FIG. 7A.

As used herein “cell penetrating peptide” abbreviated as “CPP” refers to a peptide that is capable of crossing biological membrane. Cell penetrating peptides are also called cell-permeable peptides, protein-transduction domains (PTD) or membrane-translocation sequences (MTS). CPPs have the ability to translocate in vitro and/or in vivo the mammalian cell membranes and enter into cells and/or cell nuclei, and directs a conjugated compound of interest, such as a drug or marker, to a desired cellular destination.

Several proteins and their peptide derivatives have been found to comprise cell internalization properties including but not limited to the Human Immunodeficency Virus type 1 (HIV-I) protein Tat (Ruben et al J. Virol. 63, 1-8 (1989)), the herpes virus tegument protein VP22 (Elliott and O'Hare, Cell 88, 223-233 (1997)), Penetratin (Derossi et al, J. Biol. Chem. 271, 18188-18193 (1996)), protegrin 1 (PG-I) anti-microbial peptide SynB (Kokryakov et al, FEBS Lett. 327, 231-236 (1993)) and the basic fibroblast growth factor (Jans, Faseb J. 8, 841-847 (1994)). These carrier peptides are typically highly cationic and arginine or lysine rich. Indeed, synthetic poly-arginine peptides have been shown to be internalized with a high level of efficiency (Futaki et al, J. Mol. Recognit. 16, 260-264 (2003); Suzuki et al, J. Biol. Chem. (2001)).

According to a specific embodiment, the CPP is selected from the group consisting of Human Immunodeficency Virus type 1 (HIV-I) protein Tat, the herpes virus tegument protein VP22, Penetratin, protegrin 1 (PG-I) anti-microbial peptide SynB, the basic fibroblast growth factor, synthetic poly-arginine peptide, or peptide derivative thereof possessing cell internalization properties.

According to a specific embodiment, the CPP comprises an amino acid sequence having the following formula (I):

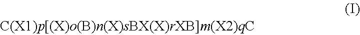

Wherein,
X1 and X2 are independently amino acid sequences of 1 to 20 amino acids;
p and q independently are whole numbers between 0 and 5, preferably 0 or 1;
B is independently a basic amino acid, X is independently a non-basic amino acid;
C is independently nothing or any moiety comprising a thioether bond linked to the remainder of the conjugate, preferably the moiety is a cysteine or cysteamine;
m is 1 or 2;
n is 1, 2 or 3;
is O or 1;
r is 0 or 1;
s is 0, 1, 2 or 3.

In a specific embodiment, the CPP is derived from human proteins, thus avoiding the immunogenicity when administered to humans.

Examples of a human CPP is LPIN3 [RRKRRRRRK, SEQ ID NO: 107 from the nuclear localization sequence (NLS) of human nuclear phosphatase, LPIN3], Hph-1-CPP (see e.g., Lim et al. Mol Cells. 2012 Dec. 31; 34(6): 577-582), Lactoferrin [hLF, see e.g., Duchardt et al. J Biol Chem. 2009 Dec. 25; 284(52): 36099-36108].

U.S. Pat. No. 9,303,076 and Table 1 therein lists numerous human CPPs and is hereby incorporated by reference in its entirety.

According to some embodiments of the invention, the peptide is selected from the group consisting of:

```
  (SEQ ID NO: 108)
GAAEAAARVYDLGLRRLRQRRRLRRERVRA;
  (SEQ ID NO: 109)
IREIME KFGKQPVSLPARRLKLRGRKRRQR;
  (SEQ ID NO: 110)
YLKVVRKHHRVIAGQFFGHHHTDSF RMLYD;
  (SEQ ID NO: 111)
SKVRFCSGRKRPVRRRPEPQLKGIVTRLFS;
  (SEQ ID NO: 112)
SMSVLEPGTAKKHKGGILRKGAKLFFRRRH;
  (SEQ ID NO: 113)
QRKIGGRGRIISPYRTPVLR RHRYSIFRST;
  (SEQ ID NO: 114)
QHVRIRVIKKKKVIMKKRKKLTLTRPTPLV;
  (SEQ ID NO: 115)
FHFFPRRPRIHFRFPNRPFVPSRCNHRFPF;
  (SEQ ID NO: 116
FALLGDFFRKSKEKIGKEFK RIVQRIKDFLRNLVPRTES.
```

According to said particular embodiment, the CPP is a peptide comprising an amino acid sequence represented by formula (I) as defined above.

In a more specific embodiment, CPPs of the present invention are also able to solubilize highly lipophilic molecules and/or to modify their pharmacokinetics and tissue distribution compared to said molecule non-conjugated to a CPP of the present invention.

In a particular embodiment, the CPP is capable of reacting in vitro and/or in vivo with cell surface glycosaminoglycans. Such CPPs were described in the PCT patent applications No WO 01/64738 and No WO 05/016960 filed by DIATOS and in De Coupade et al (Biochem J. 390:407-18 (2005), each of which is incorporated by reference in its entirety).

According to some embodiment, these peptides are amino acid sequences originating from human heparin binding proteins and/or anti-DNA antibodies selected from the group comprising: the lipoproteins such as human apolipoprotein B or E (Cardin et al, Biochem. Biosphys. Res. Com. 154:741 (1988)), the agrine (Campanelli et al, Development 122:1663-1672 (1996)), the insulin growth factor binding protein (Fowlkes et al, Endocrinol. 138:2280-2285 (1997)), the human platelet-derived growth factor (Maher et al, MoL Cell. Biol. 9:2251-2253 (1989)), the human extracellular superoxide dismutase (EC-SOD) (Inoue et al, FEBS 269:89-92 (1990)), the human heparin-binding epidermal growth factor-like growth factor (HB-EGF) (Arkonac et al, J. Biol. Chem. 273:4400-4405 (1998)), the acid fibroblast growth factor (aFGF) (Fromm et al, Arch. Biochem. Bioph. 343:92 (1997)), the basic fibroblast growth factor (bFGF) (Yayon et al, Cell 64:841-848 (1991)), the human intestinal mucin 2 sequence (Xu et al, Glyconjug J. 13:81-90 (1996)), the human gamma interferon (Lortat-Jacob & Grimaud, FEBS 280:152-154 (1991)), the subunit p40 of human interleukin 12 (Hasan et al., J. Immunol. 162:1064-1070 (1999)), the factor 1-alpha derived from stromal cells (Amara et al, J. Biol. Chem. 272:200-204 (1999)), the human neutrophil derived “heparin binding protein” (CAP 37/azurocidin) (Pohl et al, FEBS 272:200-204 (1990)), an immunoglobulin molecule such as CDR2 and/or CDR3 regions of the anti-DNA monoclonal murine antibody F4.1 (Avrameas et al, Proc. Natl. Acad. Sci. 95:5601 (1998)), the hyper variable CDR3 region of human anti-DNA monoclonal antibody R.TT79 (Stevenson et al, J. Autoimmunity 6:809 (1993)), the hyper variable area CDR2 and/or CDR3 of the human anti-DNA monoclonal antibody NE-I (Hirabayashi et al, Scand. J. Immunol. 37:533 (1993)), the hypervariable area CDR3 of the human anti-DNA monoclonal antibody RT72 (Kalsi et al, Lupus 4:375 (1995)). Each of which may present a different embodiments and the citations are incorporated by reference in their entirety.

The capacity of the CPPs to react with/bind to glycosaminoglycans (GAGs) can be determined by direct or indirect glycosaminoglycan-binding assays known in the art, such as the affinity coelectrophoresis (ACE) assay for peptide glycosaminoglycan binding described in the PCT patent application WO 00/45831. Several other methods well known in the art are available for analyzing GAG-peptides interactions, for example the method described in the PCT patent application WO 01/64738 or by Weisgraber and Rail (J. Biol. Chem., 262(33): 11097-103) (specific example with the apolipoprotein B-100); or by a modified ELISA test: 96-well plates are coated with specific GAG (chondroitin sulfate A, B and C, heparin, heparin sulfate, hyaluronic acid, keratin sulfate, syndecan), peptide conjugated to a marker is then added for a defined time; after extensive washing, peptide binding is determined using specific analysis related to the marker.

According to a specific embodiment the CPP is of any length. For example CPP is less than or equal to 100, 50, 25, 22, 10, 8, 7, 6, 5 or 4 amino acids in length. For example CPP is greater or shorter than or equal to 7, 10, 22, 25, 50, 100 amino acids in length. The suitable length and design of the CPP will be easily determined by those skilled in the art. As general references on CPPs it can be cited: CELL PENETRATING PEPTIDES: PROCESSES AND APPLICATIONS, edited by UIo Langel (2002); or Advanced Drug Delivery Reviews 57:489-660 (2005).

In specific embodiments, the CPP is 5, 6, 7, 8, 9, 10, 11, 12, 13, 14, 15, 16, 17, 18, 19, 20, 21, 22, 23, 24 or 25 amino acids in length.

According to a specific embodiment, the CPP is 7-25, 8-20, 8-19, 8-18, 8-17, 8-16, 8-15, 8-14, 8-13, 8-12, 8-11, 8-10, 8-9 amino acids in length.

According to a specific embodiment, the CPP is 9-20, 9-19, 9-18, 9-17, 9-16, 9-15, 9-14, 9-13, 9-12, 9-11, 9-10 amino acids in length.

According to a specific embodiment, the CPP is 10-20, 10-19, 10-18, 10-17, 10-16, 10-15, 10-14, 10-13, 10-12, 10-11 amino acids in length.

According to a specific embodiment, the CPP is 11-20, 11-19, 11-18, 11-17, 11-16, 11-15, 11-14, 11-13, 11-12 amino acids in length.

In a specific embodiment, the CPP comprises the amino acid sequence of formula (I) of having 7 or more amino acid in length but less than 50 amino acids in length, preferably less than 25, preferably less than 10. Generally, the amino acid sequence of formula (I) has more than 7 or 8 amino acids, preferably more than 10.

According to particular embodiments, the CPP is a peptide comprising an amino acid sequence of formula (I) wherein C is absent or is in any position of the amino acid sequence, or more preferably is at the C or N terminal position of said amino acid sequence. A moiety comprising a thiol group can be added at any position of the amino acid sequence of formula (I) to conjugate the peptide to the water insoluble drug (e.g., SN28), derivative or analog thereof, via a thioether bond. In a specific embodiment, the moiety comprising a thiol group is either at the C or N terminal position of said amino acid sequence (i.e., the moiety is present at only one of C and N terminal positions, at the other C or N position C of formula (I) is absent). Specifically, the moiety is a cysteine or cysteamine.

According to a specific embodiment, X1 and X2 independently are amino sequences from 2 to 15 amino acids (e.g., 7-15), more preferably from 7 to 10 amino acids. They can comprise either basic or non basic amino acids. More particularly, X1 and X2 are devoid of cysteine amino acid.

The term “basic amino acid” means any amino acid positively charged at pH 7, particularly any amino acid having guanidyl, amidinyl or amino moieties. The terms “guanidyl” and “guanidine” are used interchangeably to refer to a moiety having the formula —HN═C(NH₂) NH (unprotonated form). As an example, arginine contains a guanidyl (guanidino) moiety, and is also referred to as 2-amino-5-guanidinovaleric acid or a-amino-6-guanidinovaleric acid. The terms “amidinyl” and “amidino” are used interchangeably and refer to a moiety having the formula-C(═NH)(NH₂). Specific highly basic amino acids are histidine (H), arginine (R) and/or lysine (K), and more specifically K and R.

The term “non basic amino acid” means any amino acid residue not positively charged at pH 7 or below. It includes consequently, any non polar amino acid (i.e., hydrophobic amino acid), polar uncharged amino acid and negatively charged amino acid at pH 7.

As used herein non polar amino acids are A, I, L, M, F, P, W, and V. Polar uncharged amino acids are N, C, Q, G, S, T and Y. Negatively charged amino acids are D and E.

According to a specific embodiment, the non basic amino acids comprised in the BX(X)rXB moiety of formula (I) are selected in the group consisting of glutamic acid (E), glycine (G), glutamine (Q) s serine(S), threonine (T), leucine (L), valine (V), proline (P), and citrulline.

Specific amino acid sequences according to the invention are those wherein:
is 1, and/or
p and/or q is 1, and/or
XI is a sequence of 3 to 12 amino acids, and/or
X2 is a sequence of 2 to 10 amino acids, and/or
r is 0 and/or
m is 1.

Accordingly, specific CPPs derived from human heparin binding proteins and capable of specifically penetrating into a cell are selected from the group consisting of:
DPV3 (SEQ ID NO: 1): CPP reacting with heparin and dimer of a peptide derived from the C-terminal part of the sequence of human extracellular superoxide dismutase (EC-SOD) (Inoue et al, FEBS 269:89-92 (1990)).
DPV6 (SEQ ID NO: 2): CPP reacting with heparin and derived from the amino acid sequence of the C-terminal part of chain A of the human platelet-derived growth factor (Maher et al, MoI. Cell. Biol. 9:2251-2253 (1989)).
DPV7 (SEQ ID NO: 3) and DPV7b (SEQ ID NO: 4): CPPs reacting with heparin and derived from the C-terminal part of the sequence of the human heparin-binding epidermal growth factor-like growth factor (HB-EGF) (Arkonac et al, J. Biol. Chem. 273:4400-4405 (1998)).
DPV1O (SEQ ID NO: 5): CPP reacting with heparin and corresponding to the C-terminal part of the human intestinal mucin 2 sequence (Xu et al., Glyconjug J. 13:81-90 (1996)).
DPV3/10 (SEQ ID NO: 6): CPP reacting with heparin and derived from the C-terminal part of the sequence of human extracellular superoxide dismutase (EC-SOD) (see above) and from C-terminal part of the human intestinal mucin 2 sequence (see above).
DPV 10/6 (SEQ ID NO: 7): CPP reacting with heparin and derived from the C-terminal part of the human intestinal mucin 2 sequence (see above) and from the C-terminal part of chain A of the platelet-derived growth factor (see above).
DPV1047 (SEQ ID NO: 8) and DPV1048 (SEQ ID NO: 9): CPP reacting with heparin, derived from the amino acid sequence (3358-3372) of the human lipoprotein B (Cardin et al, Biochem. Biosphys. Res. Com. 154:741 (1988)) and from the sequence of the peptide corresponding to the hypervariable area CDR3 of the human anti-DNA monoclonal antibody NE-I (Hirabayashi et al, Scand. J. Immunol. 37:533 (1993)).
DPV15 (SEQ ID NO: 10) and DPV15b (SEQ ID NO: 11): CPPs reacting with heparin and containing part of the sequence of the “heparin binding protein” CAP 37.

According to the invention, the cell penetrating peptide is more specifically selected from one peptide identified in Table 1 below.

According to a specific embodiment, the CPP is DPV1047 (SEQ ID NO: 8).

TABLE 1

| SEQ | | Amino acid sequences |
|---|---|---|
| ID | Cell penetrating | (Nter to Cter) in |
| NO: | peptides | one letter code |
| 1 | DPV3 | RKKRRRESRKKRRRES |
| 2 | DPV6 | GRPRESGKKRKRKRLKP |
| 3 | DPV7 | GKRKKKGKLGKKRDP |
| 4 | DPV7b | GKRKKKGKLGKKRPRSR |
| 6 | DPV3/10 | RKKRRRESRRARRSPRHL |
| 7 | DPV10/6 | SRRARRSPRESGKKRKRKR |
| 8 | DPV1047 | VKRGLKLRHVRPRVTRMDV |
| 9 | DPV1048 | VKRGLKLRHVRPRVTRDV |
| 5 | DPV10 | SRRARRSPRHLGSG |
| 10 | DPV15 | LRRERQSRLRRERQSR |
| 11 | DPV15b | GAYDLRRRERQSRLRRRERQSR |
| 12 | Buforin II | TRSSRAGLQFPVGRVHRLLRK |
| 13 | GALA | WEAALAEALAEALAEHLAEALAEA |
| | | LEALAA |
| | Haptotactic peptides: | |
| 14 | Cβ | KGSWYSMRKMSMKIRPFFPQQ |
| 15 | preCγ | KTRYYSMKKTTMKIIPFNRL |
| 16 | CαE | RGADYSLRAVRMKIRPLVTQ |
| 17 | hCT(9-32) | LGTYTQDFNKFHTFPQTAIGVGAP |
| 18 | HN-1 | TSPLNIHNGQKL |
| 19 | Influenza virus | NSAAFEDLRVLS |
| | Nucleoprotein (NLS) | |
| 20 | KALA | WEAKLAKALAKALAKHLAKALAKA |
| | | LKACEA |
| 21 | K-FGF | AAVALLPAVLLALLAP |
| 22 | Ku70 | VPMLKPMLKE |
| 23 | MAP | KLALKLALKALKAALKLA |
| 24 | MPG | GALFLGFLGAAGSTMGAWSQPKKK |
| | | RKV |
| 25 | MPM (IP/K-FGF) | AAVALLPAVLLALLAP |
| 26 | N50 (NLS of NF-κB P50 | VQRKRQKLM |
| 27 | Pep-1 | KETWWETWWTEWSQPKKKRKV |
| 28 | Pep-7 | SDLWEMMMVSLACQY |
| 29 | Penetratin | RQIKIWFQNRRMKWKK |
| 30 | Short Penetratin | RRMKWKK |
| 31 | Poly Arginine-R7 | RRRRRRR |
| 32 | Poly Arginine-R9 | RRRRRRRRR |
| 33 | pISL | RVIRVWFQNKRCKDKK |
| 34 | Prion mouse PrPc1-28 | MANLGYWLLALFVTMWTDVGLCKK |
| | | RPKP |
| 35 | pVEC | LLIILRRRIRKQAHAHSK |
| 36 | SAP | VRLPPPVRLPPPVRLPPP |
| 37 | SV-40 (NLS) | PKKKRKV |
| 38 | SynB1 | RGGRLSYSRRRFSTSTGR |
| 39 | SynB3 | RRLSYSRRRF |
| 40 | SynB4 | AWSFRVSYRGISYRRSR |
| 41 | Tat₄₇₋₆₀ | YGRKKRRQRRRPPQ |
| 42 | Tat₄₇₋₅₇ | YGRKKRRQRRR |
| 43 | Tat₄₉₋₅₇ | RKKRRQRRR |
| 44 | Tat₄₈₋₆₀ | GRKKRRQRRRPPQ |
| 45 | Transportan | GWTLNSAGYLLGKINLKALAALAK |
| | | KIL |
| 46 | Transportan 10 | AGYLLGKINLKALAALAKKIL |
| 47 | Transportan | GWTLNSAGYLLG |
| | derivatives: | |

| | | |
|---|---|---|
| 48 | | INLKALAALAKKIL |
| 49 | VP22 | DAATATRGRSAASRPTERPRAPAR |
| | | SASRPRRPVD |
| 50 | VT5 | DPKGDPKGVTVTVTVTVTGKGDPK |
| | | PD |
| 51 | DPV51 | KRGLKLRH |

According to other embodiments the CPP is selected from the group consisting of DPV1047, N50, Short Penetratin, SV-40 (NLS), SynB3, Tat47-57, Tat49.57, DPV51 and CLIP6.

According to other embodiments the CPP is selected from the group consisting of DPV1047 and CLIP6.

According to other embodiments, the CPP candidate may be also one of the peptides described in Table 2 (Chen et al. “Heparan Sulfate-Binding Cell Penetrating Peptide for Tumor Targeting and Migration Inhibition”, BioMed Research International (2015) www(dot)dx(dot)doi(dot)org/10.1155/2015/237969), like the CPPecp—NYRWRCKNQN (animal origin, SEQ ID NO: 117), or most preferably its heparan sulfate binding sequence part (e.g., 7-15 amino acids long). For example, the TRRRERRA sequence (SEQ ID NO: 118) from the Delta N (1-22) peptide (viral origin), the NRRMKWKK s (SEQ ID NO: 119) sequence from the PDX-1-PTD peptide (animal origin), or the FRKSKEKI (SEQ ID NO: 120) sequence from the LL-37 (1-37) peptide (Antimicrobial peptide), etc.

TABLE 2

| SEQ | Peptide | | Heparan |
|---|---|---|---|
| ID | name | Sequence and predicted | sulfate |
| NO: | pI | secondary structure* | binding region |
| | Viral protein-derived CPP | | |
| 52 | TAT peptide | RKKRRQRRR | RKKRRQRR |
| | (49-57) | | |
| | pI: 12.70 | CCCCCCCCC | |
| 53 | Nucleoplasmin | KRPAAIKKAGQAKKKK | Not reported |
| | NLS (155- | | |
| | 170) | | |
| | pI: 11.47 | CcHHHHHHHhHHHhCC 55 | |
| 54 | HTLV-II Rex | TRRQRTRRARRNR | TRRQRT |
| | (4-16) | | |
| | pI: 12.85 | CCCCHHHHCCCCC | |
| 55 | Lambda-N | QTRRRERRAEKQAQW | RRRERR |
| | (48-62) | | |
| | pI: 11.83 | CCHHHHHHHHHHCCC | |
| 56 | Phi21 N (12- | TAKTRYKARRAELIAERR | KTRYKARRA |
| | 29) | | |
| | pI: 11.45 | CCCCCCCHHHHHHHHHHH | |
| 57 | Delta N (1- | MDAQTRRRERRAEKQAQ | TRRRERRA |
| | 22) | WKAAN | |
| | pI: 11.44 | CCCCHHHHHHHHHHHHH | |
| | | HHHHH | |
| 58 | FHV coat | RRRRNRTRRNRRRVR | RRRRNRTRR |
| | (35-49) | | |
| | pI: 13.00 | CCCCCCCCCCCCCCC | NRRRVR |
| 59 | BMV coat | KMTRAQRRAAARRNRW | ARRNRW |
| | (8-26) | TAR | |
| | pI: 12.78 | CcCHHHHHHHHHHhccccC | |
| 60 | HIV-1 Rev | RQARRNRRRRWR | RQARRNRRR |
| | (35-46) | | |
| | pI: 12.85 | CCCCCCCHHHHH | RWR |
| 61 | Rev (26-42) | TRQARRNRRRRWRERQF | TRQARRNRR |
| | pI: 12.54 | CCCCCCCCHHHHHHHHH | RRWRERQF |
| 62 | CPP from | ENARQGAARVTSWLGRQ | Basic residues |
| | pestivirus | LRIAGKRLEGRSKTWFG | |
| | envelope | AYA | |
| | glycoprotein | | |
| | (Erns) (194- | | |
| | 220) | | |
| | pI: 11.72 | CCCccchHHHHHHHHHHHH | |
| | | HHHHhhhCCCCccccccC | |
| 63 | gp41 fusion | GALFLGWLGAAGSTMGA | WSQPKKKR |
| | sequence | WSQPKKKRKV | |
| | pI: 11.33 | HHHHHHHHHHHHHHHHH | KV |
| | | CCCCCCCCCC | |
| 64 | VP22 | DAATATRGRSAASRPTER | SRPRRP |
| | | PRAPARSASRPRRPVD | |
| | pI: 12.10 | CCCccCCCCCCCCCCCCCC | |
| | | CCCCCCCCCCCCCCC | |
| 65 | SV40 NLS | PKKKRKV | PKKKRKV |
| | pI: 11.33 | CCCCCCC | |
| | Animal homeostatic modulator-derived CPP | | |
| 66 | Penetratin | RQIKIWFQNRRMKWKK | NRRMKW |
| | pI: 12.31 | CCCHHHHHHHCCCCCC | |
| 67 | CPPecp | NYRWRCKNQN | RWRCK |
| | pI: 10.05 | CCCCCCCCCC | |
| 68 | Apolipoprote | SVKAQYKKNSDKHRLMR | Basic residues |
| | in B binding | KRGLK | |
| | domain | | |
| | pI: 9.82 | CCcccccCCCCCCCCCCcCCC | |
| | | C | |
| 69 | hCT(9~32) | LGTYTQDFNKFHTFPQTA | Not reported |
| | | IGVGAP | |
| | pI: 6.74 | HHHHHHHHHHHHHHCHH | |
| | | HHHCCCC | |
| 70 | pVEC | LLIILRRRIRKQAHAHSK | LRRRIRK |
| | (615-632) | ChhHHHHHHHHHHHhcCC | |
| | pI: 12.48 | | |
| 71 | hLF peptide | KCFQWQRNMRKVRGPP | MRKVRG |
| | | VSCIKR | |
| | pI: 10.93 | CCCchhHHHHhCCCCCcecec | |
| | | C | |
| 72 | PDX-1-PTD | RHIKIWFQNRRMKWKK | NRRMKWKK |
| | pI: 12.31 | ChhhhHhhhhhhhhcC | |
| 73 | LL-37 (1-37) | LLGDFFRKSKEKIGKEFK | FRKSKEKI |
| | | RIVQRIKDFLRNLVPRTES | |
| | pI: 10.61 | HHHHHHHHHHHHHHHHH | |
| | | HHHHHHHHHHHHHHHCC | |
| | | CCC | |
| 74 | SynB1 (1-18) | RGGRLSYSRRRFSTSTGR | Basic residues |
| | pI: 12.30 | CCCCEEEEECCEEEEECC | |
| 75 | SynB3 | RRLSYSRRRF | Basic residues |
| | pI: 12.18 | CCCCcccCCC | |
| | Toxin-derived CPP | | |
| 76 | bPrPp (1-28) | MVKSKIGSWILVLFVAM | Basic residues |
| | | WSDVGLCKKRP | |
| | pI: 10.03 | CCCCCCCCHHHHHHHHHH | |
| | | HHHHHCCCC | |
| 77 | Crotamine | YKQCHKKGGHCFPKEKI | RWRWK |
| | (1-42) | CLPPSSDFGKMDCRWRW | |
| | | KCCKKGSG | |
| | pI: 9.51 | CCHHHHHCEEEECCCCCC | |
| | | CCCCCEECCCCCCCCCEEE | |
| | | ECCCC | |
| 78 | Maurocalcine | GDCLPHLKLCKENKDCC | SKKCKR and |
| | (MCa) | SKKCKRRGTNIEKRCR | EKRCR |
| | (1-33) | | |
| | pI: 9.46 | CCCCCCCCCCCCHHHCCC | |
| | | CEEECCCCCCCCEEE | |

*The confidence of the prediction is denoted by scaling the predictions from week (lower-case letter) to strong (upper-case letter). “H,” “E,” and “C” refer to a-helical, B-strand, and random coil propensities, respectively.

Other CPPs are listed in WO2018/111153 which is hereby incorporated by reference in its entirety.

Also included in the scope of the invention are natural and synthetic homologs (e.g., at least 80%, 81%, 82%, 83%, 84%, 85%, 86%, 87%, 88%, 89%, 90%, 91%, 92%, 93%, 94%, 95%, 96%, 97%, 98%, 99% identical to any of the peptides referred to or described herein) as determined by Blast algorithm using default parameters.

The peptides of some embodiments of the invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

The peptides of some embodiments of the invention are preferably utilized in a linear form, although it will be appreciated that in cases where cyclicization does not severely interfere with peptide characteristics, cyclic forms of the peptide can also be utilized.

According to some embodiments, the CPP is attached directly (without a linking group or a linker which are used interchangeably here) to said water insoluble drug.

According to other embodiments, the CPP is attached to said water insoluble drug via a linking group.

As used herein, the terms “linker”, “linking group” or “crosslinker” are used interchangeably herein and refer to a chain conjugating/linking two moieties together and comprising one or more atoms.

When a linker is used, the linkage may be an intermediary bonding group such as described below, all such linking groups and others described below, are hereinafter referred to as linker moieties, or are derived from the crosslinking reagents described below.

In accordance with the present invention, each CPP is linked to at least one drug moiety and more preferably to one drug moiety.

In a particular embodiment, the CPP is prepared such as to facilitate linkage to more than one drug moiety, each drug moiety being the same or different. For example, the CPP may comprise components that themselves facilitate the attachment of more than one drug moiety such as derivatives of naturally occurring amino acids, such as cysteine, or insertion of a multi-valent synthetic amino acid or a linker with multiple active sites. In this manner, a single CPP may carry between 2 and 10 moieties. In this further embodiment each drug moiety may be directly or indirectly linked to the CPP by the same or different linker moiety. When more than one different type of drug moiety is attached, it is possible to co-ordinate the ratios and dosages of the individual drugs to facilitate the administration of specific drug combinations.

Direct linkage may occur through any convenient functional group on the drug moiety such as a hydroxy, carboxy or amino group.

Indirect linkage occurs through a linking moiety. Linking moieties may also provide intramolecular flexibility or adjust intramolecular distances between conjugated domains and thereby may help preserve biological activity. Suitable linking moieties include bi and multifunctional organic radicals independently selected from substituted or unsubstituted alkyl, substituted or unsubstituted heteroalkyl, substituted or unsubstituted aryl, substituted or unsubstituted heteroaryl, aldehydes, acids, esters, anhydrides, sulphydryl or carboxyl groups, such as maleimido derivatives, maleimido cyclohexane derivatives, maleimido benzoic acid derivatives, maleimidocaproic acid derivatives and succinimido derivatives or may be derived from cyanogen bromide or chloride, succinimidyl esters or sulphonic halides and the like or combinations thereof.

Each of the above terms (e.g., “alkyl”, “heteroalkyl”, “aryl” and “heteroaryl”) include both substituted and unsubstituted forms of the indicated radical. Preferred substituents for each type of radical are provided below.

Substituents for the alkyl, and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generally referred to as “alkyl substituents” and “heteroalkyl substituents”, respectively, and they can be one or more of a variety of groups selected from, but not limited to −OR¹, =0, ═NR′, ═N-0R′, —NR′R″, —SR′, -halogen, —SiR¹R¹¹R″, —OC(O)R′, —C(O)R′, —CO₂R′, —CONR′R″, —OC(O)NR¹R″, —NR¹¹C(O)R′, —NR′—C(O)NR¹¹R″, —NR¹¹C(O)₂R¹, —NR—C(NR¹R¹¹\hat{ }NR″″, —NR—C(NR′R″)═NR^{1M}, —S(O)R′, —S(O)₂R¹, —S(O)₂NR¹R″, —NRSO₂R′, NRR⁵SO₂R″, —CN and —NO₂ in a number ranging from zero to (2m′+1), where m′ is the total number of carbon atoms in such radical. R¹, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the present invention includes more than one R group, for example, each of the R groups is independently selected as are each R¹, R″, R′″ and R″″ groups when more than one of these groups is present. When R¹ and R″ are attached to the same nitrogen atom, they can be combined with the nitrogen atom to form a 5-, 6-, or 7-membered ring. For example, NR¹R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, C(O)CH₂OCH₃, and the like).

Similar to the substituents described for the alkyl radical, the aryl substituents and heteroaryl substituents are generally referred to as “aryl substituents” and “heteroaryl substituents”, respectively and are varied and selected from, for example: halogen, —OR′, =0, ═NR¹, ═N—OR′, —NR¹R″, —SR, -halogen, —SiR¹R¹¹R″, —OC(O)R¹, —C(O)R¹, —CO₂R¹, —CONR¹R″, —OC(O)NR¹R″, —NR¹¹C(O)R′, —NR′—C(0)NR″R′″₅—NR¹¹C(O)₂R′, —NR—C(NR¹R¹O═NR″, —S(O)R′, —S(O)₂R¹, —S(O)₂NR¹R¹¹, —NRSO₂R, —CN and —NO₂, —R¹, —N₃, —CH(Ph)₂, fluoro (CᵣC₄)alkoxy, and 1 IuOrO(C₁-C₄)alkyl, in a number ranging from zero to the total number of open valences on the aromatic ring system; and where R¹, R″, R¹¹¹ and R¹¹″ are preferably independently selected from hydrogen, (C₁-C₈) alkyl and heteroalkyl, unsubstituted aryl and heteroaryl, (unsubstituted arylHQ-C\hat{ }alkyl, and (unsubstituted aryl)oxy-(C₁-C₄) alkyl. When a linking moiety of the present invention includes more than one R group, for example, each of the R groups is independently selected as are each R, R″, R′″ and R″″ groups when more than one of these groups is present.

Two of the aryl substituents on adjacent atoms of the aryl or heteroaryl ring may optionally be replaced with a substituent of the formula -T-C(O)—(CRR′)_{V}-U-, wherein T and U are independently NR—, —O—, —CRR′— or a single bond, and v is an integer of from 0 to 3. Alternatively, two of the substituents on adjacent atoms of the aryl or heteroaryl ring may optionally be replaced with a substituent of the formula -A-(CH₂)x-B—, wherein A and B are independently-CRR′—, —O—, —NR—, —S—, —S(O)—, —S(O)₂—, —S(O)₂NR— or a single bond, and x is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)b-X—(CR″R″)d-, where b and d are independently integers of from O to 3, and X is -0-, —NR′—, —S—, —S(O)—, —S(O)₂—, or —S(O)₂NR—. The substituents R, R, R″ and R″ are preferably independently selected from hydrogen or substituted or unsubstituted (C₁-C₆) alkyl.

As used herein, the term “heteroatom” includes oxygen (O), nitrogen (N), sulfur(S) and silicon (Si).

As used before, the symbol “R” is a general abbreviation that represents a substituent group that is selected from substituted or unsubstituted alkyl, substituted or unsubstituted heteroalkyl, substituted or unsubstituted aryl, substituted or unsubstituted heteroaryl, and substituted or unsubstituted heterocyclyl groups.

The functional groups {i.e. reactive groups) on the linker moiety used to form covalent bonds between linker and drugs on the one hand, as well as linker and CPP on the other hand, may be the same {i.e., homofunctional groups) or different types of functional groups {i.e., heterofunctional groups), including more particularly amino, hydrazino, hydroxyl, thiol, maleimido, carbonyl, and carboxyl groups. According to a preferred embodiment, the functional groups are selected from carboxyl (—COOH) and maleimido groups. The linker may include a short sequence of from 1 to 4 amino acid residues that optionally includes a thiol group through which the linker moiety bonds to the CPP.

In specific embodiments, coupling of the CPP and the drug moiety can be accomplished via a cross-linking reagent. There are several intermolecular cross-linking reagents which can be utilized, see for example, Means and Feeney, CHEMICAL MODIFICATION OF PROTEINS, Holden-Day, 1974, pp. 39-43. Among these reagents are, for example, iV-succinimidyl 3-(2-pyridyldithio) propionate (SPDP) or N, N¹-(1,3-phenylene) bismaleimide (both of which are highly specific for sulfhydryl groups and form irreversible linkages); N,N′-ethylene-bis-(iodoacetamide) or other such reagent having 6 to 11 carbon methylene bridges (which are relatively specific for sulfhydryl groups); and 1, 5-difluoro-2,4-dinitrobenzene (which forms irreversible linkages with amino and tyrosine groups). Other cross-linking reagents useful for this purpose include: p,p′-difluoro-N,N^{T}-dinitrodiphenylsulfone (which forms irreversible cross-linkages with amino and phenolic groups); dimethyl adipimidate (which is specific for amino groups); phenol-1,4-disulfonylchloride (which reacts principally with amino groups); hexamethylenediisocyanate or diisothiocyanate, or azophenyl-p-diisocyanate (which reacts principally with amino groups); glutaraldehyde (which reacts with several different side chains) and disdiazobenzidine (which reacts primarily with tyrosine and histidine); N-3-Maleimidopropanoic acid; N-6-Maleimidocaproic acid; N-11-Maleimidoundecanoic acid, 4-(N-maleimidomethyl)cyclohexane-1-carboxy-ó-amidocaproic acid; 4-[(N-maleimidoethyl) carboxamidoethyl (Peg) 4 carboxamidomethyl]cyclohexanecarboxy lie acid.

As mentioned before, cross-linking reagents may be homobifunctional, i.e., having two functional groups that undergo the same reaction. An example of a homobifunctional cross-linking reagent is bismaleimidohexane (“BMH”). BMH contains two maleimide functional groups, which react specifically with sulfhydryl-containing compounds under mild conditions (pH 6.5-7.7). The two maleimide groups are connected by a hydrocarbon chain. Therefore, BMH is useful for irreversible cross-linking of polypeptides that contain cysteine residues. Cross-linking reagents may also be heterobifunctional. Heterobifunctional cross-linking reagents have two different functional groups, for example an amine-reactive group and a thiol-reactive group, that will cross-link two moieties having free amines and thiols, respectively. Preferred heterobifunctional cross-linking reagents are succinimidyl 4-(N-maleimidomethyl)cyclohexane-1-carboxylate (“SMCC”), Succinimidyl-4-(N-maleimidomethyl)-cyclohexane-1-carboxy (6-amidocaproate) (“LC-SMCC”), N-maleimidobenzoyl-N-hydroxysuccinimide ester (“MBS”), and succinimide 4-(p-maleimidophenyl) butyrate (“SMPB”), an extended chain analog of MBS. The succinimidyl group of these cross-linking reagents reacts with a primary amine forming an amide bond, and the thiol-reactive maleimide forms a covalent thioether bond with the thiol group (e.g., of a cy stein).

Cross-linking reagents often have low solubility in water. A hydrophilic moiety, such as a sulfonate group, may be added to the cross-linking reagent to improve its water solubility. Sulfo-MBS and sulfo-SMCC are examples of cross-linking reagents modified for water solubility.

According to a specific embodiment, the linking group is bifunctional.

According to a specific embodiment, the linking group is cleavable.

According to a specific embodiment, the linking group is non-cleavable.

Many cross-linking reagents yield a conjugate that is essentially non-cleavable under cellular conditions. However, some cross-linking reagents contain a covalent bond, such as a disulfide, that is cleavable under cellular conditions. For example, Traut's reagent, dithiobis(succinimidylpropionate) (“DSP”), and N-succinimidyl 3-(2-pyridyldithio) propionate (“SPDP”) are well-known cleavable cross-linking reagents. Direct disulfide linkage may also be useful.

Numerous cross-linking reagents, including the ones discussed above, are commercially available. Detailed instructions for their use are readily available from the commercial suppliers. A general reference on protein cross-linking and conjugate preparation is: Wong, CHEMISTRY OF PROTEIN CONJUGATION AND CROSS-LINKING, CRC Press (1991).

The linkers that can be used according to the present invention may differ between each other by their stability in biological fluids (e.g. human plasma) when conjugated. The term “stability” is defined as the half-life time of release of the drug from the conjugate of the invention, which is dependent on the chosen linker.

According to some embodiments of the invention, the conjugate of the present invention is stable, in particular it presents a half-life time of 1, 2, 3, 4, 5, 6, 7, 8, 9, or 10 hours. An unstable conjugate will release the drug moiety with shorter half-life time, e.g. <5 minutes. A highly stable conjugate has a plasma half-life time above 11 hours. According to an embodiment, the conjugate is stable in human plasma in vitro with a half-life time of about 1 to 6.5 hours at 37° C. The half life time of the conjugate is determined using methods which are well known in the art and described in WO2007113687, which is hereby incorporated by reference in its entirety.

According to a specific embodiment, the heterobifunctional cross-linking reagents of the present invention comprise a free-COOH group and a free maleimide group. Examples of cross-linking reagents, in a non-limiting manner:
iV-3-Maleimidopropanoic acid Sold by PIERCE (Ref: 22296) And more particularly, the conjugate of the invention derives from the following crosslinking reagents:
JV-6-Maleimidocaproic acid Sold by Sigma (Ref: M8904)
Λ/-II-Maleimidoundecanoic acid sold by PIERCE (Ref: 22211)
4-(N-maleimidomethyl)cyclohexane-1-carboxy-6-amidocaproic acid
4-[(N-maleimidoethyl) carboxamidoethyl(Peg)4 carboxamidomethyl]cyclohexanecarboxylic acid
4-[(N-maleimidomethyl)cyclohexane-1-carboxy-6-amidohexanecarboxamido methyl]cyclohexanecarboxylic acid
4-[((N-maleimidomethyl)cyclohexanecarboxamido)methyl]cyclohexanecarboxylic acid Other examples of linkers, and conjugates containing same are described in WO2007113687.

As mentioned, the linker can be cleavable or non-cleavable, such as described in Table 3 below.

TABLE 3*

| Chemical | | | |
|---|---|---|---|
| trigger | Structure | Mechanism | Payload |
| Acid | Hydrazone trigger | Linkers cleavage by low pH | Calicheamicin |
| cleavable | Carbonate trigger | of tumor acidic | SN-38 |
| triggers | Silyl ether trigger | microenvironment or | MMAE |
| | | lysosomes | |
| GSH | Disulfide trigger | Linkers cleavage by high | DM1, DM3, MMAE |
| cleavable | | level of GSH in cytoplasm | PBD |
| trigger | | | |
| Fe(II) | 1,2,4-Trioxolane | Linker cleavage by | MMAE |
| cleavable | trigger | elevating levels of ferrous | |
| trigger | | iron | |
| Cathepsin | Dipeptide trigger | Linkers cleavage by | MMAE, DM1 |
| cleavable | Triglycyl (CX) | cathepsin in lysosomes | DM1 |
| triggers | trigger | | |
| | cBu-Cit trigger | | MMAE, PBD |
| Glycosidase | β-Glucuronide | Linkers cleavage by β- | MMAE |
| cleavable | trigger | glucuronidase in lysosomes | |
| triggers | β-Galactoside | Linkers cleavage by β- | MMAE |
| | trigger | galactosidase in lysosomes | |
| Phosphatase | Pyrophosphate | Linkers cleavage by | Budesonide |
| cleavable | trigger | phosphatase and | |
| triggers | | pyrophosphates in | |
| | | lysosomes | |
| Sulfatase | Arylsulfate trigger | Linkers cleavage by | MMAE |
| cleavable | | sulfatase in lysosomes | |
| trigger | | | |
| Photo- | Heptamethine | Linkers cleavage by | CA-4 |
| responsive | cyanine | irradiation with NIR light | |
| cleavable | fluorophore | (λ = 650-900 nm) | |
| triggers | trigger | | |
| | O-Nitrobenzyl | Linkers cleavage by | MMAE |
| | trigger | irradiation with UV light | |
| | | (λ = 365 nm) | |
| | PC4AP trigger | Linkers cleavage both by | DOX |
| | | irradiation with near- | |
| | | infrared (NIR) light | |
| | | (λ = 365 nm) and | |
| | | intramolecular addition | |
| | | reaction with nearby amine | |
| Bioorthogonal | dsProc trigger | Linkers cleavage by the | DOX |
| cleavable | | bioorthogonal cleavage | |
| trigger | | pair: Cu(I)-BTTAA/dsProc | |
| Non-cleavable | MD linker | No linker cleavage, ADCs | TRMRA |
| linkers | PEG linkers with | metabolizes amino acid | PBD Dimer |
| | intermediates of | appendage, a linker and | |
| | alkyne, triazole | molecule cytotoxicity upon | |
| | and piperazine | entry lysosome | |
| | Mal-PAB linker | | MMAE |

*(taken from Z. Su et al. Acta Pharmaceutica Sinica B, 2021, which is hereby incorporated by reference in its entirety):

According to other embodiments the linker and/or CPP is such that is described in FIG. 8.

The drug molecule (SN-38) has 2 hydroxyl groups that may be anchoring points for connecting the linker molecule (Scheme 1, shown in red and blue, FIG. 8).

The introduction of the standard linker (Linker 1, Scheme 2 of FIG. 8) to the blue hydroxyl group results in the formation of an ester group that is typically more stable than a carbonate group (Linker 2, Scheme 2). Linker 1 has a maleimide group which is an ideal handle for the introduction of a CPP at this position. The MW of this linker is: 359 daltons.

Interestingly, linker 2, that is attached to the red hydroxyl group forms a carbonate group, that is 5 times more stable than the ester group at the same position (S-J Moon et al., J. Med. Chem., 2008, 51, 6916-6926). Linker 2 has the same maleimide group for introducing a variety of CPPs. MW of linker 2:1234 daltons; note that this linker has a MW that is over 3-fold higher than linker 1. Both linkers are ideal for attaching several CPPs.

As linker 2 is prepared by click chemistry, there is a possibility of synthesizing a similar linker that has a thiol group instead of a maleimide group (linker 3, FIG. 8). This would allow to attach the CPP to the linker via a disulfide bond.

As the levels of GSH in the NGs are negligible, this could be a nice addition to the project. At this point, however, I would recommend to stick to linkers 1 and 2 and to vary the CPPs.

According to a specific embodiment, the CPP is selected from Table 4 below.

TABLE 4

| | CPP sequence | | Net | |
|---|---|---|---|---|
| | with dC handle/ | | positive | |
| CPP | SEQ ID NO: | MW | charge | Notes |
| SV-40 | dCPKKKRKV 101 | 986 | 5 | This CPP has |
| (NLS) | | daltons | | been |
| | | | | extensively |
| | | | | studied in a |
| | | | | variety of drug |
| | | | | delivery |
| | | | | systems |
| AIP6 | dCRRLRWR 102 | 1045 | 4 | anti- |
| | | daltons | | inflammatory |
| | | | | activity² |
| Tat49-57 | dCRKKRRQRRR | 1443 | 8 | High net |
| | 103 | daltons | | positive |
| | | | | charge |
| CPPecp | dC(Trt)RYRWRC | 1527 | 4 | Trt and Acm |
| | (Acm)KNQN 104 | dlatons | | are orthogonal |
| | | | | PGs^{3,} ⁴ |
| CLIP6 | dCKVRVRVRVdP | 2334 | 8 | Uptake |
| | PTRVRERVK | daltons | | mechanism is |
| | 105 | | | diffusive (non- |
| | | | | endosomal)⁵ |

²Y. F. Wang et al., Mol. Therapy, 2011, 19(10), 1849-1857.
³J. Spears et al., Chem. Soc. Rev., 2021, 50, 11098-11155.
⁴The Trt group is removed during SPS (Fmoc) and the Acm is stable. The terminal Cys then may be attached to the linker via a disulfide bond. The final step is to remove the Acm protection group from the central Cys with PdC12 and DTT.
⁵H. Medina et al., Angew. Chem. Int. Ed., 2016, 55, 3369-3372.

The present invention also relates to methods for preparing the conjugates (an embodiment of which is provided in the Examples section which follows).

Thus, the conjugate of the invention may be prepared by any method known in the art.

For example, the CPP (or peptide) can be prepared using conventional solution- or solid-phase peptide synthesis methods. This peptide can then be reacted directly with the drug moiety, a suitable reactive derivative of a drug moiety, or a cross-linking reagent.

A drug moiety or a derivative thereof may be attached to the CPP through e.g. thioether, hydrazone, amide, ester, ether, carbamate, thiocarbamate or disulphide bond formation.

Alternatively, a linker group, as described above is introduced by reaction of a cross-linking reagent, with an appropriate function of the CPP, in particular a thiol group, followed by formation of a covalent bond between the linker group and the drug moiety.

Multivalent drug-delivery conjugates may be obtained, inter alia, by successive extension of an appropriate function of the CPP with, for instance, bivalent or trivalent chemical groups.

According to another preferred embodiment, the cross-linking reagent is coupled to the drug moiety prior to reaction with the CPP.

Using these methods, the skilled person will be capable of preparing a wide variety of drug-carrier conjugates utilizing a variety of linker moieties. An appropriate group on the drug moiety may be selected for attachment to the CPP and if desired a linker joined to the drug or CPP, or both prior to their coupling. Alternatively, drug may also be modified so as to allow conjugation.

According to a specific embodiment the linking group is 4-{4-[(N-maleimydomethyl)cyclohexanecarboxamido]methyl}cyclohexane-1-carboxylic acid linker (BCH) especially when the peptide is DPV1047 and the drug is SN38.

According to an aspect of the invention, there is provided a method of producing a composition comprising a water insoluble drug, the method comprising:
(a) subjecting cells to a hypotonic treatment so as to obtain swollen intact cells;
(b) subjecting the swollen intact cells to flow shearing to obtain ruptured cells while avoiding nuclei lysis;
(c) filtering the ruptured cells to obtain a cell preparation devoid of nuclei;
(d) subjecting the cell preparation devoid of nuclei to purification by anion exchange chromatography, affinity chromatography, and/or filtration by size, to obtain ghosts, wherein the ghosts can be subjected to a further step of downsizing the ghosts using a high shear homogenizer or microfluidizer or extruder to obtain spherical particles of 35-400 nm;
(e) purifying the ghosts or spherical particles;
(f) incubating the spherical particles with a conjugate comprising a water insoluble drug linked to a cell penetrating peptide (CPP) under conditions which allow encapsulation of the conjugate in the particles; and
(g) removing free conjugate not undergoing the encapsulation.

Thus, the cells as described above, may be cultured in order to obtain enough cells from which spherical particles can be produced in a large-scale manner.

According to a specific embodiment, the amount of cells for starting the process of producing the spherical particles is at least, 1×10⁶, 0.5×10⁷, 1×10⁷, 0.5×10⁸, 1×10⁸, 0.5×10⁹, 1×10⁹, 1×10¹⁰, 1×10¹¹, 1×10¹².

Methods of culturing cells are well known in the art.

The selection of the media and settings are at the discretion of the skilled artisan.

Typically, in order to obtain large amounts of cells, the cells are grown in suspension although two dimensional settings are also contemplated herein. The cells can be used fresh or after thawing a frozen batch.

As used herein “a suspension culture” refers to a culture in which the cells are grown free floating (not attached to any matrix) or, when adherent, attached to solid carriers such as microcarriers or beads and grown in suspension. Such carriers can be composed of macromolecules such as cellulose, dextran, agarose, or acrylamide, or inorganic materials such as glass. The surfaces of the carriers may be further modified by physical or chemical treatments, such as adsorption or covalent cross-linking of molecular entities with a desired charge or other desired characteristic. Alternatively, the carrier may consist of a cell or cells encapsulated within a matrix that allows perfusion of sub-cellular sized material. Microcarrier culture has significant advantages, including the scale-up of cultures, and also allows cell units to be conveniently exposed to selected culture conditions as required in order to cause the desired cellular process. In the broadest embodiment, therefore, the invention provides a method for culturing cells in vitro, comprising growing said cells adhered to a microcarrier or bead.

According to a specific embodiment, the cells are selected having a population doubling level (PDL) which would increase yield of spherical particles.

According to a specific embodiment, the expansion is carried out following thawing a working cell batch and expansion under xeno-free growth conditions in a controllable bioreactor. Such conditions can be further scaled-up such as to a 50 L or larger reactors (e.g., of Biostat STR® reactors). It has already been demonstrated that MSCs can successfully be grown on microcarriers in single-use stirred bioreactors at benchtop-scale instead of the commonly used planar, one- or multiple-layer flasks, such as CellSTACKs or Cell Factories.

The cultivation process of cells, e.g., MSCs in large scale setting typically includes 3 steps: (1) initial cell attachment, (2) cell expansion, and (3) cell harvest. The microcarriers along with medium are transferred to the bioreactor (e.g., Univessel® SU) and equilibrated at 37° C. Dynamic conditions are then applied, e.g., agitation, shaking. Afterwards, the thawed and pooled MSCs are inoculated at an initial density at the range of 10³ cells cm⁻². No agitation is performed for a while e.g., 2-6 h, to allow the cells to attach to the microcarriers. The reactor is then filled with medium to the maximal working volume. When present, the impeller speed is accelerated according to the discretion of the skilled artisan e.g., initially set to X e.g., 100 rpm and increased to Y e.g., 135 rpm as cultivation progresses. MSCs, for example, can be grown at 37° C., pH 7.2 and 0.1 vvm headspace aeration for a predetermined number of days, e.g., 7 days. On a predetermined day of cultivation it is possible to perform at least 50% medium exchange or by adding a fed batch to prevent nutrient limitation. Once the maximum cell density is reached (e.g., day 7 of cultivation), the cells are separated from the microcarriers such as by using a sieving procedure combined with enzymatic cell (e.g., TrypLE™ Select) detachment and washing steps. Finally, the microcarrier-free suspension is centrifuged followed by supernatant removal and cell resuspension in fresh culture medium before initiation of NGs process or alternatively vialing and freezing occurs, if so desired. An exemplary embodiment is provided in the Examples section which follows.

As used herein “population doubling level (PDL)” refers to the total number of times the cells in a given population have doubled during in vitro culture.

According to a specific embodiment, the cells (e.g., MSCs) exhibit a PDL of 15-30, e.g., 15-27, 15-25, 15-22, 15-20.

According to a specific embodiment, the cells (e.g., MSCs) exhibit a PDL of 18-20.

Cells, e.g., MSCs, post harvesting and prior to particle production, are characterized by flow cytometry for expression of typical markers e.g., MSCs markers and for no expression of negative hematopoietic markers (e.g., as per ISCT guidelines).

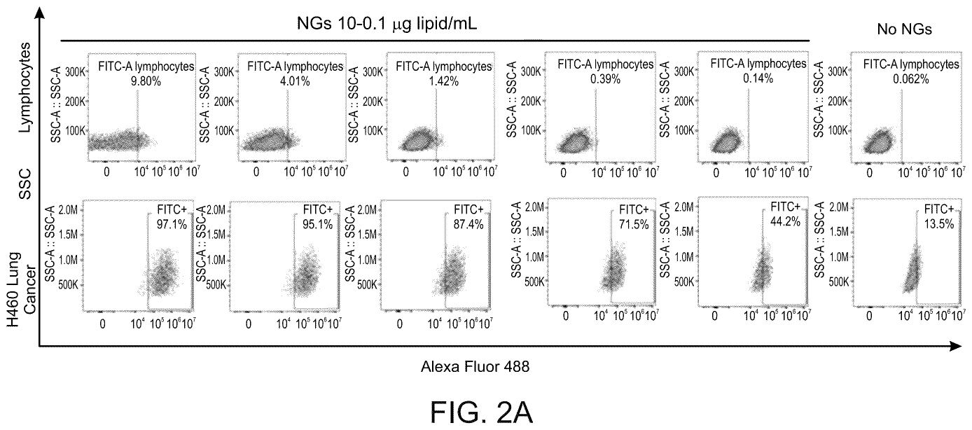

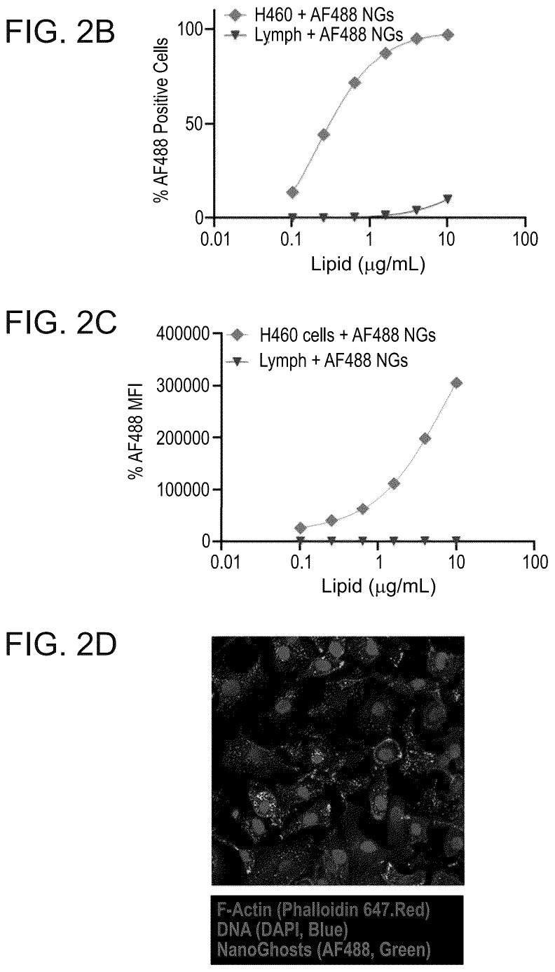

The spherical particles-derived therefrom can be evaluated by determining marker expression, e.g., MSCs surface marker expression (see above), lipid and/or protein content and/or functionally such as by particle uptake by cancer cells, see e.g., FIGS. 2A-D. Such qualifications may help in determining the most effective culturing and production methods.

Once harvested, the cells can be used fresh or subjected to freezing procedures to be used later on (as a single batch or pooled).

According to a specific embodiment, the cells are of a single cell type, meaning that the population is pure [>95% of the cells in culture are of a single cell type, e.g., ≥95% MSCs (as can be assessed by CD73+/CD90+/CD105+ signature)].

It will be appreciated that the present teachings do not necessitate the step of culturing the cells, but rather, the present method of producing spherical particles can be initiated with a cell preparation following culturing.

According to some embodiments, the method comprises culturing the cells prior to step (a) which is a hypotonic treatment.

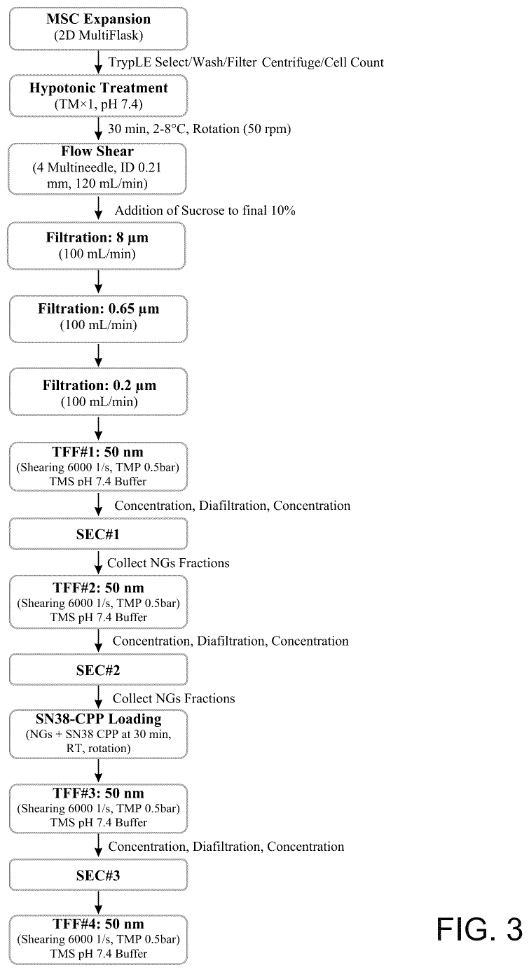

Embodiments of the method are presented in FIG. 3.

As mentioned, a cell pellet (following centrifugation) can be subjected to hypotonic treatment to obtain swollen intact cells.

Hypotonic treatment may be the first step in the downstream processing of the cells to spherical particles. This step is intended to swell/enlarge cells and ease their shear-mediated breakdown in the following production step.

As used herein “hypotonic treatment” refers to incubating the cells in a solution that has lower osmotic pressure than that of the cells.

Measures are taken to achieve maximal cell swelling while avoiding cell membrane rupture.

According to a specific embodiment, the hypotonic treatment is done under dynamic conditions, e.g., under rotation (e.g., 10-100 rpm).

According to specific embodiments, the treatment is effected for 10-60 minutes, e.g., 5-40 minutes e.g., 20-40 minutes, 25-35 minutes.

According to a specific embodiment, the treatment is performed under 2-8° C.

According to a specific embodiment, the osmolarity of the hypotonic treatment is at least 5 mOsm/kg, e.g., 5-100 mOsm/kg, e.g., 5-20 mOsm/kg.

According to a specific embodiment, the hypotonic solution is Tris Magnesium™×1 pH 7.4. Other Examples include but are not limited to PBS, Hepes, NaPi or MgSO4, each having the indicated osmolarity.

Osmolarity can be assessed by an osmometer (e.g., 3320 Single-Sample Micro Osmometer, Advanced Instruments).

The cell's diameter and/or cell viability assays can be determined to evaluate the effect of the hypotonic treatment. For example, treatment for 15 min at 2-8° C. resulted in cell swelling to a diameter of 29.7±2.6 μm (assessed by FlowCam 400, Fluid Imaging Technologies), similar to the diameter of the cells post incubation with DDW (29.7±3.0 μm).

According to a specific embodiment, the hypotonic treatment results in cell swelling of at least about 20%, 30%, 40%, 50%, 60%, 70%, 80%, 90%, 1.5 folds, 2 folds, 5 folds or more, as determined by cell diameter.

Following hypotonic treatment, the cells are subjected to rupturing using flow shearing. This is in stark contrast to traditional protocols for producing cell derived liposomes as in WO2011/024172, that make use of a homogenizer and then extruder that breaks the nuclei (see also FIG. 3). The gentle shear force of flow shearing allows release/detachment of the cell membrane without nuclei lysis. Nuclei can then be filtered out without jeopardizing lipid yield.

Thus, the swollen cells are subjected to flow shearing to obtain ruptured cells while avoiding nuclei lysis.

As used herein “flow shearing” refers to mechanical cell membrane breakage by shear forces. The advantage of this method is that it releases the intracellular content and especially the nuclei in an intact form and as such prevents the contamination of the sample with DNA.

Typically, this is done by using nanoscaled obstacles in the form of a needle, channel or tube. It is also possible to make use of a capillary pump in the lysis region to accelerate the flow and reach higher shear forces.

Avoiding nuclei lysis can be qualified by determining nuclei markers such as lamin. If no nuclei are damaged, the nucleus protein marker is not detected following flow shearing and nuclei filtration.

Hence, according to some embodiments of the method, there is no use of a homogenizer at this stage, or a sonicator.

According to a specific embodiment, the flow shearing is done with a needle/tube/channel characterized by an internal diameter (ID) of 100-400 μm and/or a needle/tube/channel length of 2-50 mm (e.g., 0.21 mm internal diameter (ID) needle, 13 mm length).

In order to improve efficacy, this step can be done using a multi-channel/needle/tube device.

The ruptured cells are filtered to obtain a preparation which is devoid of nuclei. Typically, a filter with a cut-off of, 1.2-10 μm is used, e.g., 3-10 μm, 3-8 μm, 3-9 μm, e.g., 8 μm. Such a cutoff would ensure depletion of nuclei i.e., over 90% of the nuclei.

Another step of filtration with a smaller cut-off can be employed to remove other intracellular organelles such as mitochondria, ER, Golgi and the like. In such a case a filter cut-off of 0.45-1.19 μm, 0.45-1.1 μm, 0.45-0.85 μm is used, e.g., 0.65 μm.

As opposed to the teachings of WO2011/024172 which are shown in FIG. 3, here, the filtrate (supernatant) is collected while the pellet is discarded. In FIG. 3, clearly the nuclei are present at this stage because the pellet is collected by centrifugation.

Following filtration, the cell preparation devoid of nuclei is subjected to size separation.

At this stage, the preparation can also be referred to as “ghosts”, as it comprises cell membranes essentially without the intracellular organelle content.

There are numerous purification methods which can be used. These include, but are not limited to, size-exclusion chromatography (SEC), anion exchange, affinity purification, filtration, flow field-flow fractionation and deterministic lateral displacement (DLD) pillar arrays.

According to a specific embodiment, the size separation is performed by a size exclusion chromatography (SEC).

Size exclusion chromatography (SEC) is a well-established technique used for macromolecule separation based on their molecular size or hydrodynamic volumes. A typical SEC system comprises a porous stationary phase for chromatographic separation with or without coupling to a pump for elution. The commonly used stationary phase materials for membrane isolation and separation are cross-linked agarose beads (commercially named as Sepharose® (CL-2B and CL-4B) and Sephacryl® S-400). Examples of commercially available columns include, but are not limited to, IZON® qEV column, Sepharose® CL-2B column, Sepharose® CL-4B column, Sephacryl® S-500 column, Sephacryl® S-1000 column and Superdex® 200 column. Custom-made column can be used too.

According to another specific embodiment, the size separation is performed by Tangential Flow Filtration (TFF) also known as cross-flow filtration. This is an ultrafiltration (UF) technique widely used for vesicle (EV) isolation and separation. In TFF, a stream of fluids containing ghosts flow tangentially by moving across the UF membrane (hollow fiber membrane) but not directly through the membrane. Molecules smaller than molecular weight cut-off (MWCO) travel through the membrane and are discarded, while molecules larger than the cut-off level, such as ghosts, remain on the membrane and are recirculated and concentrated. TFF is more beneficial compared to conventional filters in which the fluid flows directly through the membrane, which often results in cake formation that clogs the pores. In comparison to SEC, TFF also concentrates ghosts, while SEC dilutes the isolates, making TFF suitable for large-scale ghosts isolation from diluted samples.

The TFF can be conducted using any suitable TFF device or system. For example, TFF can be conducted using a TFF system that comprises a feed reservoir, a filter device and a collection device, the feed reservoir is in fluid communication with the filter device via an inlet on the filter device, the filter device is in fluid communication with the collection device via a permeate outlet on the filter device, and the filter device is in fluid communication with the feed reservoir via a retentate outlet on the filter device.

The filter device can be in any suitable form. For example, the filter device can be in a form of a cartridge, a cassette, or a column containing a hollow fiber filter. The filter device can comprise a filtration membrane having any suitable pore size. For example, the filter device can comprise a filtration membrane having a pore size ranging from about 100 KDa to about 300 KDa, e.g., at about 100 KDa, 150 KDa, 200 KDa, 250 KDa, 300 KDa, 750 KDA, 50 nm or 100 nm or a subrange thereof.

The TFF can be conducted via any suitable type of process. For example, the TFF can be conducted via a diafiltration process. In some embodiments, the diafiltration process is a continuous, discontinuous, or sequential diafiltration process. TFF can be conducted via any suitable number of cycle(s). In some embodiments, the TFF is conducted via a single cycle. In some embodiments, the TFF is conducted via multiple cycles of diafiltration processes, e.g., multiple cycles of continuous diafiltration processes.

The present process or the TFF can further comprise collecting the ghosts. The ghosts can be collected using any suitable device, procedure or means. For example, the ghosts can be collected via a retentate outlet on the filter device.

The TFF can be conducted for any suitable purpose(s). See for example, Cells 2018, 7, 273; doi: 10.3390/cells7120273

The TFF can also be used to concentrate and/or enrich the ghosts. In some embodiments, the TFF is used to concentrate and/or enrich the nanoparticle(s) from about 2 fold to about 1000 folds, e.g., to concentrate and/or enrich the nanoparticle(s) by 2 folds, 5 folds, 10 folds, 50 folds, 100 folds, 200 folds, 300 folds, 400 folds, or a subrange thereof.

The step of removing or reducing the level of the water-miscible solvent and the step of concentrating the ghosts can be conducted as a combined step or as separate steps.

The process can be further tuned to obtain optimal TFF operating conditions (feed cross flow rate and trans membrane pressure/TMP) for maximal flux through the membrane (to minimize process time and/or membrane area and optimize pump size).

At this stage the ghosts are optionally subjected to downsizing to nanometer scale. Accordingly, the method includes downsizing (or particulating, generating particles) the ghosts using a high shear homogenizer or a microfluidizer or extruder to obtain particles which are on average 35-400 nm.

Any suitable high shear fluid processor can be used. For example, the high shear fluid processor can be a microfluidizer (or a microfluidizer processor) or a homogenizer that generates high shear force such as Benchtop GEA XStream Lab High Shear Homogenizer (with GEA NiSoX-Valve technology). Above 250 bar, particles could pass through a 0.2 μm filter allowing sterility of the end product. Importantly, up to 1,000 bar, potency of the product was unchanged, and a marginal change was observed at 1,500 bar.

In some embodiments, the high shear fluid processor used in the present process is a microfluidizer (or a microfluidizer processor). Any suitable microfluidizer (or a microfluidizer processor) can be used. In some embodiments, the microfluidizer is configured to generate a substantially constant pressure from about 10,000 psi to about 30,000 psi, e.g., at about 10,000 psi, 15,000 psi, 20,000 psi, at about 25,000 psi, 30,000 psi, or a subrange thereof.

In some embodiments, the microfluidizer has the microfluidics reaction technology (MRT) configuration that comprises, from upstream to downstream, an inlet for inputting the nanoparticle, an intensifier pump for generating a static pressure, an impinging jet chamber for generating a high shear pressure on the nanoparticle, and an outlet for outputting the nanoparticle. In some embodiments, the microfluidizer comprises a Z-type of interaction chamber. In some embodiments, the microfluidizer comprises a Y-type of interaction chamber.

The present process can further comprise cooling the preparation. In some embodiments, the spherical particles in a channel after exiting a chamber of the microfluidizer are cooled by a product chiller that contains a coolant. The product chiller or the coolant in the product chiller can be set at any suitable temperature. For example, the product chiller or the coolant in the product chiller can be set at a temperature ranging from about 2° C. to about 8° C.

According to a specific embodiment, following high shear homogenization (e.g., at 100-250 bar) the preparation is subjected to another round of TFF (e.g., at 20-100 nm, e.g., 50 nm) or SEC or anion exchange to remove residual free protein, lipid RNA or non-encapsulating payload.

An optional step of purification can be employed such as by filtering said particles to obtain spherical particles such as with at a filter cut-off of 0.2-0.5 μm, e.g., 0.2-0.22 μm.

Purification steps can be done using methods which are well known in the art, including, but not limited to TFF, size exclusion chromatography, affinity chromatography and/or anion exchange.

Once the spherical particles are formed (i.e., with or without a pharmaceutical agent, as further described hereinbelow), they may be characterized for their size distribution, composition, concentration, zeta potential, electrical surface potential, surface (local) pH, protein to lipid ratio, marker expression, and therapeutic efficacy in vitro and in vivo.

According to some embodiments of the invention, empty spherical particles or spherical particles comprising one or more pharmaceutical agent of the present invention are in the size range of 10-1000 nm, 30-500 nm, 35-400 nm, 50-200 nm, 70-150 nm.

For example, the spherical particles can have an average diameter from about 10 nm to about 1000 nm. In certain embodiments, the diameter of the nanoparticle is at least about, 20 nm, 30 nm, 40 nm, 50 nm, 60 nm, 70 nm, 80 nm, 90 nm, 100 nm, 110 nm, 120 nm, 130 nm, 140 nm, 150 nm, 200 nm, 300 nm, 400 nm, 500 nm, 600 nm, 700 nm, 800 nm, 900 nm and not exceeding 1000 nm, 900 nm, 800 nm, 700 nm, 600 nm 500 nm or any combination thereof which is considered a specific embodiment, e.g., 20-500 nm or any sub-range within about 20 nm to about 1000 nm, e.g., any range between any two of the above sizes.

An advantage of spherical particles smaller or about 100-nm is their ability to penetrate through very narrow blood vessels which is of great significance in diagnostic and treatment.

Any method known in the art can be used to determine the size of the liposome. For example, a Nicomp Submicron Particle Sizer (model 370, Nicomp, Santa Barabara, Calif.) utilizing laser light scattering can be used. Other methods of measuring liposome size include flow cytometry, photocorrelation spectroscopy, laser diffraction, low-angle laser light scattering (LALLS), medium-angle laser light scattering (MALLS), light obscuration methods (Coulter method, for example), rheology, or microscopy (light or electron).

Other methods and devices for determining size of the spherical particles include, but are not limited to, ExoView™ R100 NanoSight from NanoView (size 50-200 nM), Flow nanoAnalyzer from NanoFCM (size, concentration, phenotyping), or qNano Gold from iZon Tunable Resistive Pulse Sensing (TRPS; Size, Concentration, Charge), or Zeta View ParticleMetrix (NTA, fNTA; concentration, size, charge, phenotype).

The specific average effective particle size depends on factors such as the intended route of administration, formulation, solubility, toxicity and bioavailability of the compound.

Values of Zeta potential in experimentally tested spherical particles are provided infra.

According to some embodiments of the invention, the average zeta potential of the spherical particles (without PEGylation or pharmaceutical agent) is (−5) to (−) 50 mV, e.g., about −30 mV.

The particles can be further qualified using methods which are well known in the art. For example, as shown in the Examples section, spherical particles derived from MSCs are characterized by flow cytometry for expression of MSC identity markers as well as markers that are found expressed on their surface using Ab array.

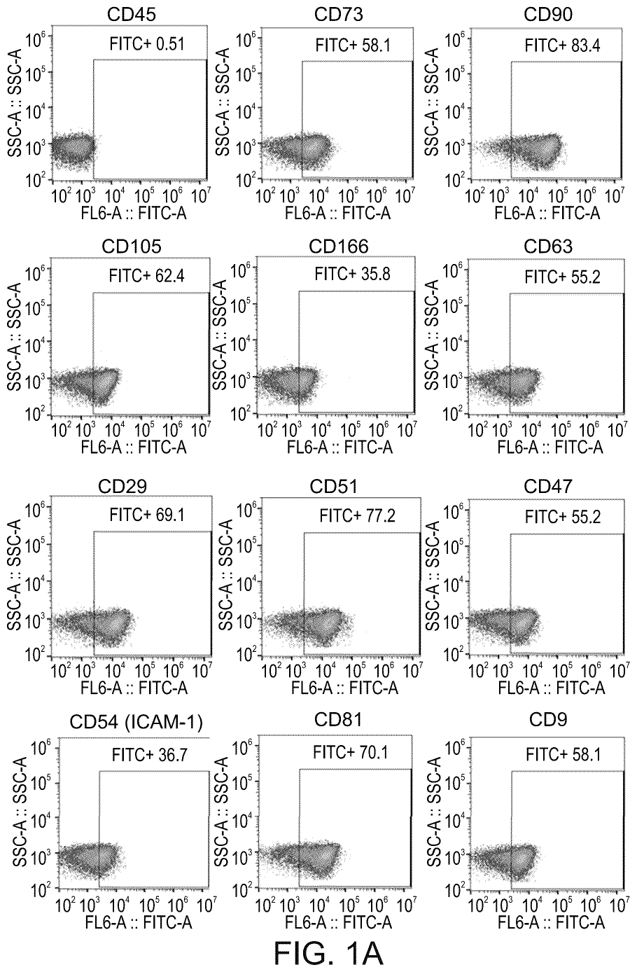

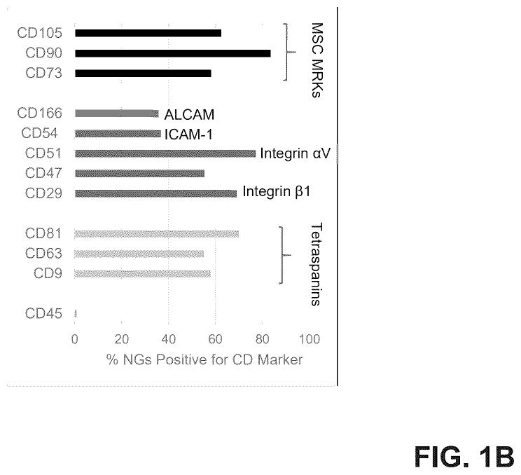

As shown in FIG. 1B, spherical particles derived from MSCs express the MSC marker CD90, CD73 and CD105 as well as other surface markers such as CD166, CD63, CD29, CD51, CD47 CD81, CD9 and ICAM-1.

According to an aspect of the invention, there is provided a composition comprising a plurality of spherical particles composed of a whole cell membrane fraction, wherein the spherical particles exhibit native membrane symmetry and expression of native markers obtainable according to the method as described herein.

According to other embodiments, there is provided a composition comprising a plurality of spherical particles composed of a whole cell membrane fraction, wherein the spherical particles exhibit native membrane symmetry and expression of native markers and wherein said plurality of spherical particles encapsulate a conjugate comprising a water insoluble drug linked to a cell penetrating peptide (CPP) and wherein the composition is obtainable according to the method as described herein.

It will be appreciated that some conjugate may also be attached (e.g., non-covalently adsorbed) to the outer surface of the particles even after purification of the particles.

According to a specific embodiment, the composition is characterized by a membrane to nucleus marker ratio higher by at least 1.5 fold, 1.6 fold, 1.7 fold, 1.8 fold, 1.9 fold, 2.0 fold, 2.2 fold, 2.5 fold, 2.7 fold, 3.0 fold, 3.5 fold, 4.0 fold, 5.0 fold, 7.0 fold, 10 fold, 20 fold (e.g., 2-5 fold) than that obtained when using a homogenizer instead of flow shearing to rupture cells, or when collecting the centrifugal pellet following homogenization instead of the filtrate following the flow shearing.

Once purified spherical particles are obtained (e.g., see FIG. 3 following TFF2 and SEC2) the method comprises incubating the spherical particles with the conjugate under conditions which allow encapsulation of the conjugate in the particles;

According to some embodiments of the invention, a ratio of said spherical particles lipid to said conjugate in said incubating is about 10:1 to 1:10, 8:1 to 1:8, 7:1 to 1:7, 6:1 to 1:6, 5:1 to 1:5, 4:1 to 1:4, 3:1 to 1:3, 2:1 to 1:2, mass ratio.

According to some embodiments of the invention, a ratio of said spherical particles and said conjugate in said incubating is about 2:1 to 1:2.

According to some embodiments of the invention, a ratio of said spherical particles and said conjugate in said incubating is about 1:1.

According to some embodiments of the invention, said conditions comprise 5-60 minutes of incubation.

According to some embodiments of the invention, said conditions comprise and 4-37° C.

Any non-encapsulated pharmaceutical agent, is removed by size separation (e.g., TFF, SEC, as described above and in FIG. 3).

According to a specific embodiment, the loading capacity of the conjugate or the drug (e.g., when the CPP is cleaved|) is at least 10%, 20%, 30%, 40%, 50%, 60%, 70%, 80%, 90% or more.

The compositions thus obtained, are of high quality in terms of purity (e.g., level of DNA in the preparation) and as such can be beneficially used in the clinic.

These can be further formulated-frozen or used fresh.

According to a further aspect of the invention, the composition has a therapeutic property per se (especially when produced from MSCs which are known for their immunomodulatory properties), but addition of a pharmaceutical agent increases its efficacy and alternatively or additionally shifts its therapeutic value per the desired activity.

As mentioned, spherical particles of the present invention are advantageously used in the clinic.

Thus, according to an aspect of the invention there is provided a method of delivering a pharmaceutical agent, the method comprising administering to a subject in need thereof the above-describe spherical particles, wherein the pharmaceutical agent is enclosed therein or adsorbed thereon, thereby delivering the pharmaceutical agent.

According to an aspect the pharmaceutical composition is for use in treating cancer.

According to an aspect of the invention there is provided a method of treating cancer in a subject in need thereof comprising administering to the subject the pharmaceutical composition as described herein thereby treating cancer.

According to an embodiment, the cells are target cells and the spherical particles contain targeting moieties, either chemically conjugated, heterologously added, as described above, or natively presented in the membranes from which the spherical particle is comprised (e.g., as in MSCs, which migrate to tumor cells).

The cell source for the spherical particles may be autologous or non-autologous (e.g., allogeneic, xenogeneic) to the subject.

The “target cell” referred to herein is a cell or a cluster of cells (of homogenous or heterogeneous population) and/or tissue to which a substance is to be delivered by using the spherical particle. Examples thereof include cancer cells, vascular endothelial cells of angiogenic cancer tissues, cancer stem cells, interstitial cells of cancer tissues, cells affected by genetic abnormality, cells infected by a pathogen and the like. The “target molecule” may be any molecule presented on the surface of the target cells. Another form of the target molecule includes molecules which are released from cells. Examples thereof include extracellular matrix components, secretions or architectures of cancer cells or interstitial cells of cancer tissues, and specific examples thereof include tumor markers, structures between cells and the like.

Delivering can be for diagnostic reasons (e.g., the spherical particle includes a diagnostic agent) or for treating (i.e., as a drug delivery tool, delivering a therapeutic agent) such as for treating cancer.

Examples of cancer include but are not limited to carcinoma, lymphoma, blastoma, sarcoma, and leukemia. Particular examples of cancerous diseases but are not limited to: Myeloid leukemia such as Chronic myelogenous leukemia. Acute myelogenous leukemia with maturation. Acute promyelocytic leukemia, Acute nonlymphocytic leukemia with increased basophils, Acute monocytic leukemia. Acute myelomonocytic leukemia with eosinophilia; Malignant lymphoma, such as Burkitt's Non-Hodgkin's; Lymphoctyic leukemia, such as Acute lumphoblastic leukemia. Chronic lymphocytic leukemia; Myeloproliferative diseases, such as Solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, skin, prostate, and ovarian.

According to a particular embodiment, the cancer is a solid tumor.

The spherical particles may be administered to the subject per se, or as part of a pharmaceutical composition.

As used herein a “pharmaceutical composition” refers to a preparation of one or more of the active ingredients described herein with other chemical components such as physiologically suitable carriers and excipients. The purpose of the pharmaceutical composition is to facilitate administration of the active ingredients to the subject.

Herein the term “active ingredient” refers to the therapeutic agent (with or without the spherical particle) accountable for the biological effect. It is to be appreciated that the spherical particle per se may have immunomodulatory function such as when prepared from membranes of MSCs or other immunomodulatory cells (e.g., immune B and T lymphocytes etc.). It is also to be appreciated that the spherical particle per se may have a cytoxoic effect on the target cells as due to membrane fusion with target cells and consequent disruption to cell membrane, cytoskeleton and functions. In such a case measures are taken to include a targeting moiety such that the cytotoxic effect becomes specific.

Hereinafter, the phrases “physiologically acceptable carrier” and “pharmaceutically acceptable carrier” which may be interchangeably used refer to a carrier or a diluent that does not cause significant irritation to the subject and does not abrogate the biological activity and properties of the administered active ingredients. An adjuvant is included under these phrases.

Herein, the term “excipient” refers to an inert substance added to the pharmaceutical composition to further facilitate administration of an active ingredient of the present invention or to increase shelf-life stability. Examples, without limitation, of excipients include calcium carbonate, calcium phosphate, various sugars and salts and types of starch, cellulose derivatives, gelatin, vegetable oils, EDTA, EGTA, Poly-L-Lysine, polyethyleneimine, Polybrene (hexadimethrine bromide), polyethylene glycols and other poly or single anions. The pharmaceutical composition may advantageously take the form of foam, aerosol or a gel.

Techniques for formulation and administration of drugs may be found in “Remington's Pharmaceutical Sciences,” Mack Publishing Co., Easton, PA, latest edition, which is incorporated herein by reference.

Suitable routes of administration include any of various suitable systemic and/or local routes of administration.

Suitable routes of administration may, for example, include the inhalation, oral, buccal, rectal, transmucosal, topical, transdermal, intradermal, transnasal, intestinal and/or parenteral routes; the intramuscular, subcutaneous and/or intramedullary injection routes; the intrathecal, direct intraventricular, intravenous, intraperitoneal, intranasal, and/or intraocular injection routes, Catheterization with or without angio balloons; and/or the route of direct injection into a tissue region of the subject.

The pharmaceutical composition may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer.

For transmucosal administration, penetrants appropriate to the barrier to be permeated are used in the formulation. Such penetrants are generally known in the art.

For oral administration, the pharmaceutical composition can be formulated readily by combining the active ingredients with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active ingredient doses.

Pharmaceutical compositions which can be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration via the inhalation route, the active ingredients for use according to the present invention can be delivered in the form of an aerosol/spray presentation from a pressurized pack or a nebulizer with the use of a suitable propellant, e.g., a fluorochlorohydrocarbon such as dichlorodifluoromethane, trichlorofluoromethane, dichloro-tetrafluoroethane; carbon dioxide; or a volatile hydrocarbon such as butane, propane, isobutane, or mixtures thereof. In the case of a pressurized aerosol, the dosage unit may be determined by providing a valve to deliver a metered amount. Capsules and cartridges of, e.g., gelatin for use in a dispenser may be formulated containing a powder mix of the active ingredients and a suitable powder base such as lactose or starch.

The pharmaceutical composition may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

A pharmaceutical composition for parenteral administration may include an aqueous solution of the active ingredients in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or spherical particles. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredients may be in powder form for constitution with a suitable vehicle, e.g., sterile, pyrogen-free water based solution, before use.

The pharmaceutical composition may also be formulated in rectal compositions such as suppositories or retention enemas, using, e.g., conventional suppository bases such as cocoa butter or other glycerides.

The pharmaceutical composition should contain the active ingredients in an amount effective to achieve disease treatment.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture and in vivo assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma or brain levels of the active ingredients which are sufficient to achieve the desired therapeutic effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of the composition to be administered will be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredients. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Materials and Methods

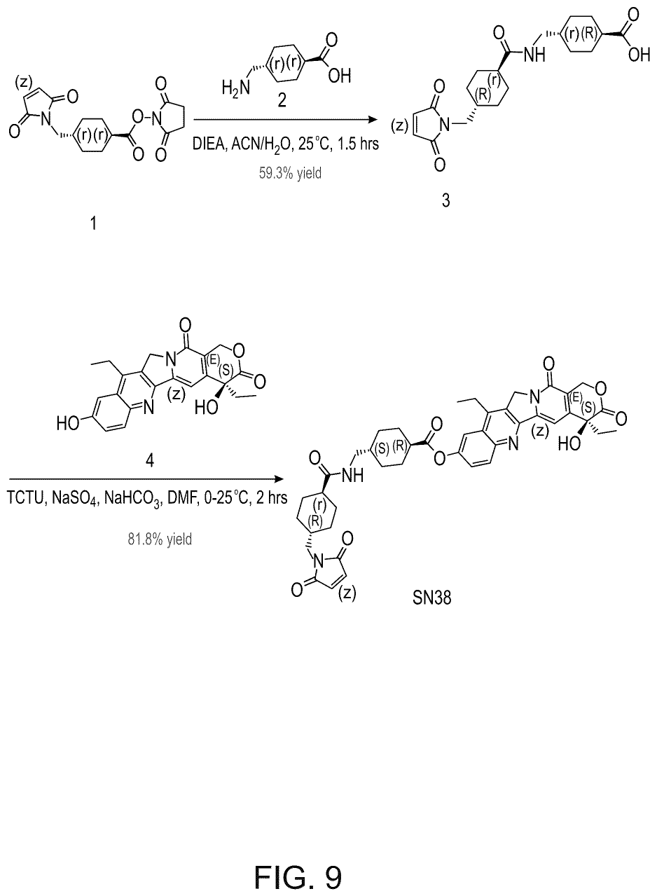

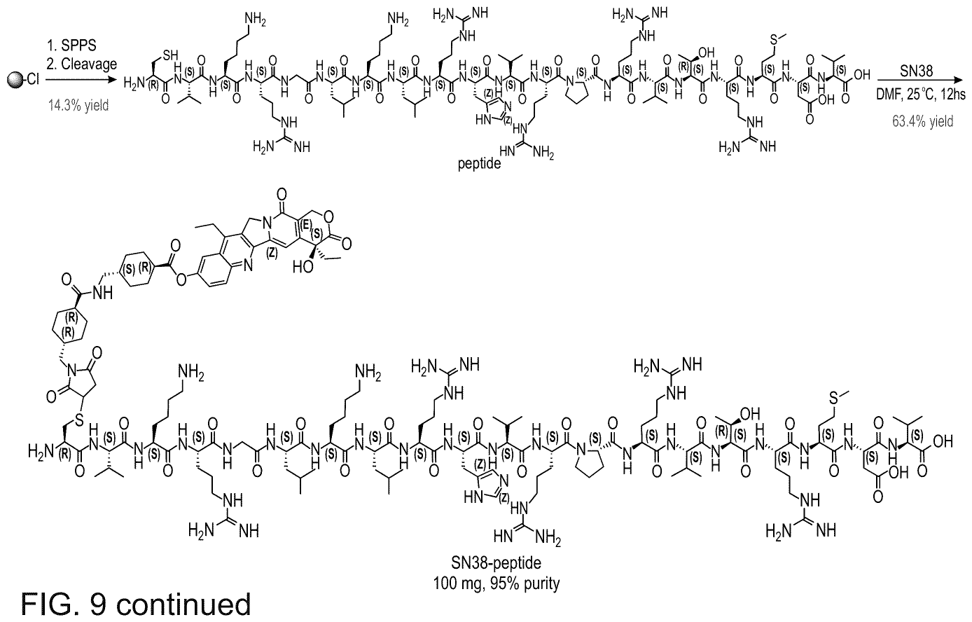

Synthesis of SN38-CPP conjugate (Drug/Peptide ratio of 1:1) is carried out as briefly described in FIG. 9 and below. To a solution of compound 1 (5.00 g, 14.9 mmol, 1.00 eq) and compound 2 (2.94 g, 18.7 mmol, 1.25 eq) in ACN (50.0 mL) and H₂O (25.0 mL) was added DIEA (483 mg, 3.74 mmol, 651 μL, 0.25 eq). The mixture was stirred at 25° C. for 1.5 hr. LCMS (EC6909-12-P2A) showed compound 1 was consumed completely and one main peak with desired mass was detected. Diluted the reaction mixture with MTBE and stirred for 5 minutes, filtered the solution and washed with MTBE (2×30 ml). The resulting white solid was dissolved in 20% MeOH/DCM. The organic phase was separated, washed with brine 100 mL (50.0 mL×2), dried over with Na₂SO₄, filtered and concentrated under reduced pressure to give a residue. Slurry the solid in acetone at ambient temperature for 30 min, filter the solid and wash with acetone to get Compound 3 (3.34 g, 8.87 mmol, 59.3% yield) as a white solid which was confirmed by LCMS (EC6909-12-P1ACHU, Rt=0.339 min, MS cal.: 367.20, MS observed: [M+H]+=377.0) and HNMR (EC6909-12-p2b).

LCMS of monitoring: EC6909-12-P2A, Rt=0.353 min, MS cal.: 367.20, MS observed: [M+H]+=377.0.

LCMS: EC6909-12-P1ACHU, Rt=0.339 min, MS cal.: 367.20, MS observed: [M+H]+=377.0.

1H NMR: EC6909-12-p2b 400 MHz, DMSO-d6

δ: 11.25-12.66 (m, 1H), 7.64 (br t, J=5.60 Hz, 1H), 7.01 (s, 2H), 3.23 (d, J=7.00 Hz, 2H), 2.85 (t, J=6.24 Hz, 2H), 2.04-2.17 (m, 2H), 1.86 (br d, J=10.6 Hz, 2H), 1.68 (br d, J=10.8 Hz, 4H), 1.59-1.65 (m, 2H), 1.18-1.18 (m, 1H), 1.15-1.34 (m, 5H), 0.83-0.93 (m, 4H).

To a solution of compound 4 (2.00 g, 5.10 mmol, 1.00 eq), compound 3 (2.11 g, 5.61 mmol, 1.10 eq), Na₂SO₄ (2.03 g, 14.2 mmol, 1.45 mL, 2.80 eq) and TCTU (2.08 g, 5.86 mmol, 1.15 eq) in DMF (40.0 mL), NaHCO₃ (1.50 g, 17.8 mmol, 693 μL, 3.50 eq) was added in one portion and the mixture was allowed to stir at 0° C. Then mixture was stirred at 25° C. for 2 hrs. LCMS (EC6909-14-P1A1) showed compound 4 was consumed completely and one main peak with desired mass was detected. The reaction mixture was diluted with 0.08 M HCl and stirred for 5 minutes. The solution was filtered and washed with MTBE (2×30.0 ml). The resulting white solid was dissolved in DCM. The organic phase was separated, washed with 0.01 M HCl (100 mL) and Brine 100 mL (50.0 mL×2), dried over with Na₂SO₄, filtered and concentrated under reduced pressure to give a residue. The solid was re-dissolved in DCM, the reaction mixture diluted with MTBE and stirred for 5 minutes. The solution was filtered and washed. The filter-cake with MTBE (2×30.0 ml), under reduced pressure to give SN38 (3.13 g, 4.17 mmol, 81.8% yield) was obtained as a pale yellow solid which was confirmed by LCMS (EC6909-14-P1ACHUN, Rt=0.469 min, MS cal.: 750.3, MS observed: [M+H]+=751.1.) and HPLC(EC6909-14-P1CCHUN, Rt=3.178 min).

LCMS of monitoring: EC6909-14-P1A1, Rt=0.470 min, MS cal.: 750.3, MS observed: [M+H]+=751.1.

LCMS: EC6909-14-P1ACHUN, Rt=0.469 min, MS cal.: 750.3, MS observed: [M+H]+=751.1.

HPLC: EC6909-14-P1CCHUN, Rt=3.178 min, 97.7% purity.

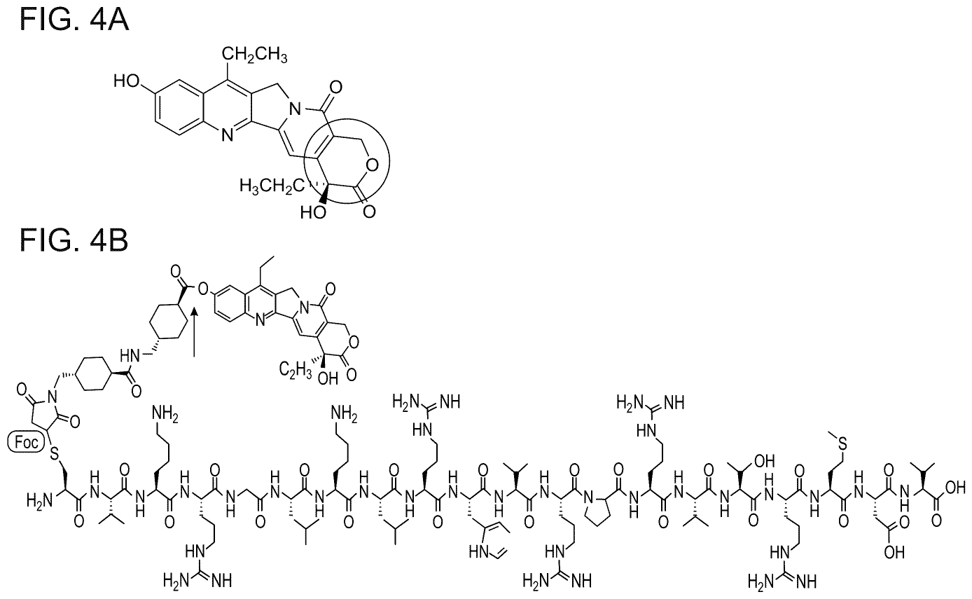

Production of SN38-loaded NGs is carried out as briefly described in FIG. 3 and below. This protocol is based on studies that tested the optimal conditions for loading NGs with SN38 that is conjugated via a linker to a 20 amino acid cell penetrating peptide (FIG. 4A-B).

NGs production was carried out using ˜3.5E9 human bone marrow mesenchymal stem cells (MSCs)/process run. MSCs used in NGs production process were thawed and expanded under xeno-free growth conditions in T225 (Passage 3) and then in CellSTACK10 (Passage 4). Cells were harvested using TrypLE Select, washed, filtered through 100 μm Sartopure PP3 capsule size 4 filter, centrifuged, counted (using NC-200 cell counter) and resuspended in hypotonic buffer.

Hypotonic Treatment: Cells (5e6 MSCs/mL cold (2-8° C.) Tris Magnesium×1 pH 7.4 buffer) underwent hypotonic treatment for 30 min at 2-8° C. under rotation (50 rpm).

Cell Breakdown by Flow Shear: Following hypotonic treatment, MSCs underwent cell breakdown by flow shear through a 4 Channel Multi Needle (4MN) device (27G ID=0.21 mm, 5 mm long) using the TFF peristaltic pump with tubing ⅛″ at a flow rate of 120 mL/min. 60% sucrose solution was added to cells homogenate to reach 10% final concentration and then the suspension was filtered through Capsule Size 4 filters as described below.

Filtration: Flow sheared suspension was filtered through:
8 μm Sartopure PP3 capsule size 4 filter at 100 mL/min (to get rid of cell nuclei)
0.65 μm Sartopure PP3 capsule size 4 filter at 100 mL/min (to get rid of large intracellular particles).
0.2 μm Sartoguard PES capsule size 4 filter at 100 mL/min (to sterile filter small sized vesicles).
Tangential Flow Filtration (TFF) #1, MicroKros 190 cm² 0.05 μm, at flow rate of 265 mL/min for shearing rate of 6000 l/s, and TMP 0.5 bar (to further purify cell derived vesicles from cell derived proteins). Material was concentrated 10-fold and then purified by diafiltration with 20 diavolumes of Tris Magnesium Sucrose (TMS)×1 pH7.4 buffer (20 times the volume of concentrated suspension, to eliminate intracellular proteins). NGs were further concentrated (˜2-4-fold), to undergo size exclusion chromatography.

Size Exclusion Chromatography (SEC) and TFF: TFF filtered material underwent SEC using IZON qEV SEC35 columns. Fractions positive for NGs were collected and underwent additional cycle of TFF and SEC.

Loading of SN38-CPP: NGs from SEC #2 were diluted to 50 ug lipid/mL in TMS pH 7.4 buffer and mixed at different Lipid to Peptide mass ratios with SN38-CPP conjugate, for 30 min at RT (in orbital shaker).

Size Exclusion Chromatography (SEC) and TFF: Loaded NGs were concentrated and purified by TFF and further purified from unloaded SN38-CPP by SEC. Fractions positive for loaded SN38 (280 nm, 620 nm and Ex/Em 380/570 nm for SN-38 fluorescence) were collected. Loaded NGs from the third SEC were concentrated by TFF, aliquoted and stored at −80° C.

Note that this process did not make use of the size reduction step, that will be integrated in the future, to allow higher lipid yield and a more consistent particle size.

Marker assessment-Antibodies (5-20 μL, according to manufacturer's instructions) were diluted to 150 μL in PBS and filtered through a 20 nm syringe filter. NGs (5-10 μL, ˜5 ug lipid) were added to 50 μL of each filtered antibody solution and incubated at RT for 30 min in the dark. Immunostained NGs were purified using ExoSpin columns (or other SEC column) and eluted with 6×100 μL with PBS. Purified NGs (˜1 μg/mL) were acquired using the CytoFlex flow cytometer under parameters suitable for detection of nanoparticles. Measurement optimization was done using non stained NGs as a reference and CD45 stained nanoghosts as negative control.

In Vitro Assessment of SN38-NGs IC50: Proliferation assay was carried out using H460 NSCLC cells (2,000 cells/well) for 72 hrs. Cells were incubated with NGs loaded with the SN38-peptide conjugate (DTS108) at a drug range of 0.3-2000 nM, or with the controls (free SN-38, free SN-38 peptide conjugate (DTS108), free peptide (DPV1047) and free NGs) for 2 hrs and 6 hrs. At 2 hrs and 6 hrs, cells were washed, fresh growth media was added, and cells were incubated for 72 hrs. Cell proliferation was measured using an ATPlite kit. IC50 was calculated for each tested material as a function of exposure time using Prism.

In Vivo Efficacy of SN38-NGs: Male Balb/c nude mice aged 5-7 weeks were implanted (using a 23 gauge needle) onto the flank with HCT116 colorectal cancer cells (5E6 cells in Matrigel 1:1). Tumor volumes measured using electronic callipers three times per week starting 7 days after implant. When tumors reached approximately 100 mm³ animals were assigned to the following treatment groups to ensure an equal spread of tumor volumes. Treatment was given IV at 200 μL per dosing occasion.

TABLE 5

| Group | N | Treatment | Dose |
|---|---|---|---|
| 1* | 8 | Control 1: Vehicle | NA; TMS |
| | | | BID, QOD |
| 2* | 8 | Control 2: NGs | 2 mg Lipid/kg |
| | | | BID, QOD |
| 3 | 8 | Control 3: SN-38 linked to a | 80 mg/kg |
| | | CPP (DTS108) | 3 times a week |
| 4* | 8 | Control 4: DTS108 in the same | 0.708 mg/kg |
| | | amount as in NGs | BID, QOD |
| 5* | 8 | NGs loaded with DTS108 - | 2 mg Lipid/kg |
| | | Loading dose 1 | BID, QOD |
| | | | 0.2 mg/kg DTS108 |
| 6* | 8 | NGs loaded with DTS108 - | 2 mg Lipid/kg |
| | | Loading dose 2 | BID, QOD |
| | | | 0.5-0.7 mg/kg DTS108 |

*Mice were fasted overnight prior to each injection
Duration of dosing: 20 days

### Example 1

### Characterization of MSC-Derived NGs

NGs were assessed for the presence of CD45 (negative control), CD9, CD29, CD47, CD51 (integrin αV), CD54 (ICAM-1), CD63, CD73, CD81, CD90, CD105 and CD166 on the NGs and the results are shown in FIGS. 2A-B.

### Example 2

### SN38 Solubilization

Since SN-38 is a highly hydrophobic drug that is not soluble in water-based solutions, several strategies were explored to increase SN-38 solubility, while ensuring no leakage post loading into NGs.

The selected strategy which showed the best results was solubilizing SN-38 via conjugation to a protein or a peptide.

A 20 amino acid cell penetrating peptide (CPP) (CVKRGLKLRHVRPRVTRMDV, SEQ ID NO: 121) (FIG. 4B) known to solubilize SN38 (solubility of >500 mg/mL in water), was used as the peptide of choice to test the strategy of solubilizing SN38 to a peptide. This selection was based on the cell penetrating characteristics of the peptide, its high-water solubility, as well as preclinical anti-tumor activity and safety in a phase 1 study (Meyer-Losic et al., 2008 and Coriat et al., 2016).

### Example 3

Loading Studies with SN-38 Peptide Conjugate

### In Vitro and In Vivo Efficacy Studies

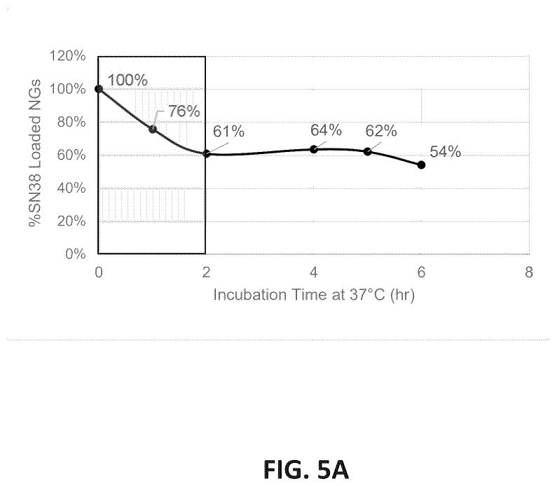

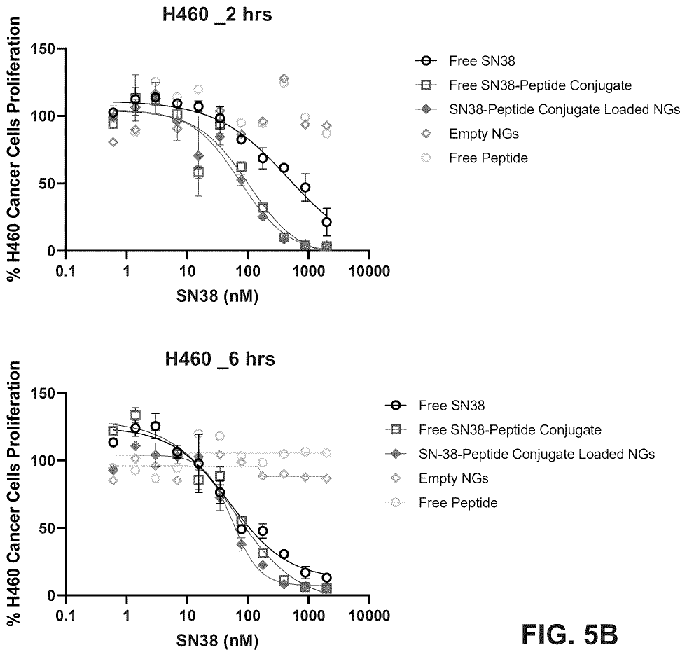

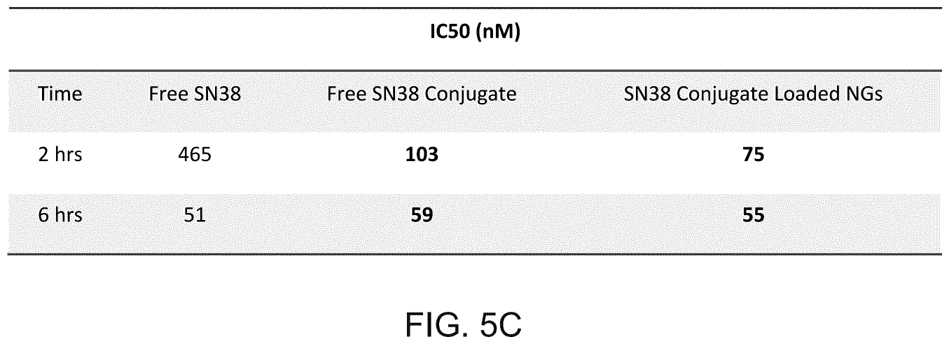

Using the SN38-CPP conjugate (Drug/Peptide ratio of 1:1) as a model solubilizing peptide, the present inventors were able to load ˜0.77 ug peptide (˜0.095 ug SN-38) per 1 ug NGs lipid under loading conditions that included a mixing ratio of 1:1 between NGs lipid and SN38-CPP conjugate (each at 50 μg/mL) in TMS (10 mM Tris, 1 mM Magnesium Chloride, 10% Sucrose, pH 7.4) for 30 min under rotation at RT (Table 1). Using the optimal loading conditions, in vitro analysis with NGs that were loaded with this SN38-CPP conjugate demonstrated the feasibility of this strategy, showing IC50 that is comparable to free SN38 and leakage profile (burst release followed by ˜4 hrs of no drug release) that supports in vivo applicability (FIGS. 5A and 5B).

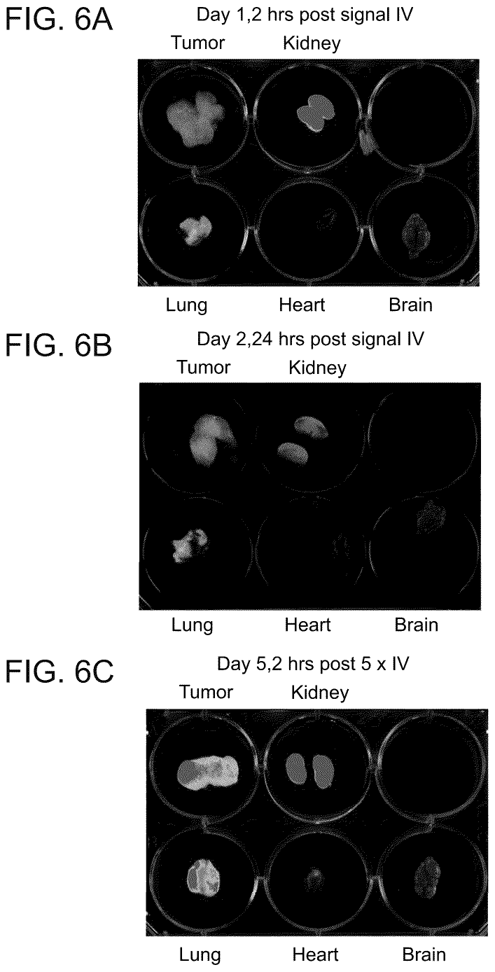

In vivo analysis with SN38-CPP conjugate was tested in the HCT116 cancer model, in which the present inventors first demonstrated biodistribution of Cy7 labeled NGs. As shown in FIG. 6A-C, Cy7 labeled NGs were biodistributed to the HCT116 CRC tumor that was implanted subcutaneously onto the flank of male balb/c nude mice, and the level of NGs in the tumor increased across the 5 daily injection days. Using the same dose that was used in the biodistribution study (2 mg NGs lipid/kg), a regiment of 2 daily injections (BID) of 2 mg SN38-CPP loaded NGs/kg was given every other day (QOD). SN38-CPP loaded NGs produced according to FIG. 3 and loaded with SN38-CPP conjugate at two loading ratios of 1:0.5 and 1:0.75, were injected to mice. Loading ratios of 1:0.5 and 1:0.75 allowed administration of a total of ˜0.2 mg/kg SN38-CPP and ˜0.5-0.7 mg/kg SN38-CPP, respectively, in every injection of 2 mg/kg NGs. As shown in FIG. 7A, SN38-CPP loaded NGs given at the two SN38-CPP loading levels, reduced tumor volume by 37-44%, while the free SN38-CPP at 80 mg/kg (given 3 times a week) reduced tumor volume by 52% and free SN-38 CPP at 0.7 mg/kg (the level given by NGs) reduced tumor volume by 12% (FIG. 7A). In addition, SN38-CPP at 80 mg/kg resulted in reduction of body weight, while SN38-CPP loaded NGs did not affect mice weight (FIG. 7B). These data give a good proof of concept (PoC) for the ability of NGs to target SN38 to the tumor using significantly lower drug levels, that eliminate drug toxicity.

TABLE 6
Optimization of SN38-CPP Loading Conditions

Loading

| Mixing Ratio | | | | NGs | NGs |
|---|---|---|---|---|---|
| (NGs Lipid/ | | Mixing | | Lipid to | Lipid to |
| SN38-CPP) | Buffer | Time | | SN38-CPP | SN38 |
| 1:1 | TM* | 30 | min | 1:0.59 | 1:0.073 |
| | TMS** | | | 1:0.68 | 1:0.084 |
| | PBS | | | 1:0.32 | 1:0.034 |
| 1:0.2 | TMS | 30 | min | 1:0.04 | 1:0.005 |
| 1:0.4 | | | | 1:0.17 | 1:0.02 |
| 1:0.7 | | | | 1:0.49 | 1:0.06 |
| 1:1 | | 7.5 | min | 1:0.57 | 1:0.07 |
| | | 15 | min | 1:0.51 | 1:0.063 |
| | | 30 | min | 1:0.53 | 1:0.065 |
| 1:0.5 | | 30 | min | 1:0.35 | 1:0.043 |
| | | 120 | min | 1:0.21 | 1:0.026 |
| 1:1 | | 30 | min | 1:0.77 | 1:0.095 |
| | | 120 | min | 1:0.66 | 1:0.082 |

*TM, Tris MgCl₂ buffer, pH 7.4;
**TMS, TM containing 10% Sucrose.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A composition comprising a plurality of spherical particles composed of a whole cell membrane fraction, wherein the spherical particles exhibit native membrane symmetry and expression of native markers and wherein said plurality of spherical particles encapsulate a conjugate comprising a water insoluble drug linked to a cell penetrating peptide (CPP).

2. The composition of claim 1, wherein said whole cell membrane faction is of mesenchymal stem cells (MSCs).

3. The composition of claim 1, wherein said water insoluble drug is selected from the group consisting of Camptothecin analogs, Auristatin derivatives, Maytansinoids derivatives, Anthracyclines, Platins, Taxanes, Gemcitabine, Calicheamicin, Camptothecin and water-insoluble PARP inhibitors.

4. The composition of claim 1, wherein said water insoluble drug is SN38.

5. The composition of claim 1, wherein said water insoluble drug is SN38 and said CPP is DPV1047.

6. The composition of claim 1, wherein said CPP is attached to said water insoluble drug via a linking group, optionally wherein said linking group is 4-{4-[(N-maleimydomethyl)cyclohexanecarboxamido]methyl}cyclohexane-1-carboxylic acid linker (BCH).

7. The composition of claim 4, wherein said linking group is bifunctional.

8. The composition of claim 4, wherein said linking group is cleavable.

9. The composition of claim 4, wherein said linking group is non-cleavable.

10. The composition of claim 1, wherein said CPP is attached directly to said water insoluble drug.

11. The composition of claim 1, wherein said CPP is 7-25 amino acids in length.

12. The composition of claim 1, wherein said CPP comprises basic amino acids.

13. The composition of claim 1, wherein said CPP is of a human protein or a homolog or ortholog thereof.

14. The composition of claim 1, wherein said CPP is derived from a glucose amine glycan binding protein, optionally wherein said CPP is derived from a heparin binding protein.

15. The composition of claim 1, wherein said CPP is selected from the group consisting of SEQ ID NO: 107-116, or, wherein said CPP is selected from the group consisting of DPV1047, N50, Short Penetratin, SV-40 (NLS), SynB3, Tat47-57, Tat49-57, DPV51 and CLIP6.

16. The composition claim 1, wherein said CPP comprises at least one synthetic amino acid and/or wherein a backbone of said CPP comprises at least one modification.

17. A method of producing a composition comprising a water insoluble drug, the method comprising:
(a) subjecting cells to a hypotonic treatment so as to obtain swollen intact cells;
(b) subjecting said swollen intact cells to flow shearing to obtain ruptured cells while avoiding nuclei lysis;
(c) filtering said ruptured cells to obtain a cell preparation devoid of nuclei;
(d) subjecting said cell preparation devoid of nuclei to purification by anion exchange chromatography, affinity chromatography, and/or filtration by size, to obtain ghosts, wherein said ghosts can be subjected to a further step of downsizing said ghosts using a high shear homogenizer or microfluidizer or extruder to obtain spherical particles of 35-400 nm;
(e) purifying said ghosts or spherical particles;
(f) incubating said spherical particles with a conjugate comprising a water insoluble drug linked to a cell penetrating peptide (CPP) under conditions which allow encapsulation of said conjugate in said particles; and
(g) removing free conjugate not undergoing said encapsulation.

18. The method of claim 17, wherein a ratio of said spherical particles lipid and said conjugate in said incubating is about 10:1 to 1:10, 2:1 to 1:2 or 1:1 mass ratio.

19. The method of claim 17, wherein said conditions comprise 5-60 minutes of incubation and/or said conditions comprise and 4-37° C.


SEQUENCE LISTING (121 sequences)

SEQ ID NO: 1 - protein, 16 aa, synthetic construct
  REGION 1..16: note = cell penetrating peptide (CPP)
RKKRRRESRKKRRRES

SEQ ID NO: 2 - protein, 17 aa, synthetic construct
  REGION 1..17: note = cell penetrating peptide (CPP)
GRPRESGKKRKRKRLKP

SEQ ID NO: 3 - protein, 15 aa, synthetic construct
  REGION 1..15: note = cell penetrating peptide (CPP)
GKRKKKGKLGKKRDP

SEQ ID NO: 4 - protein, 17 aa, synthetic construct
  REGION 1..17: note = cell penetrating peptide (CPP)
GKRKKKGKLGKKRPRSR

SEQ ID NO: 5 - protein, 18 aa, synthetic construct
  REGION 1..18: note = cell penetrating peptide (CPP)
RKKRRRESRRARRSPRHL

SEQ ID NO: 6 - protein, 19 aa, synthetic construct
  REGION 1..19: note = cell penetrating peptide (CPP)
SRRARRSPRESGKKRKRKR

SEQ ID NO: 7 - protein, 19 aa, synthetic construct
  REGION 1..19: note = cell penetrating peptide (CPP)
VKRGLKLRHVRPRVTRMDV

SEQ ID NO: 8 - protein, 18 aa, synthetic construct
  REGION 1..18: note = cell penetrating peptide (CPP)
VKRGLKLRHVRPRVTRDV

SEQ ID NO: 9 - protein, 14 aa, synthetic construct
  REGION 1..14: note = cell penetrating peptide (CPP)
SRRARRSPRHLGSG

SEQ ID NO: 10 - protein, 16 aa, synthetic construct
  REGION 1..16: note = cell penetrating peptide (CPP)
LRRERQSRLRRERQSR

SEQ ID NO: 11 - protein, 22 aa, synthetic construct
  REGION 1..22: note = cell penetrating peptide (CPP)
GAYDLRRRERQSRLRRRERQSR

SEQ ID NO: 12 - protein, 21 aa, synthetic construct
  REGION 1..21: note = cell penetrating peptide (CPP)
TRSSRAGLQFPVGRVHRLLRK

SEQ ID NO: 13 - protein, 30 aa, synthetic construct
  REGION 1..30: note = cell penetrating peptide (CPP)
WEAALAEALAEALAEHLAEALAEALEALAA

SEQ ID NO: 14 - protein, 21 aa, synthetic construct
  REGION 1..21: note = cell penetrating peptide (CPP)
KGSWYSMRKMSMKIRPFFPQQ

SEQ ID NO: 15 - protein, 20 aa, synthetic construct
  REGION 1..20: note = cell penetrating peptide (CPP)
KTRYYSMKKTTMKIIPFNRL

SEQ ID NO: 16 - protein, 20 aa, synthetic construct
  REGION 1..20: note = cell penetrating peptide (CPP)
RGADYSLRAVRMKIRPLVTQ

SEQ ID NO: 17 - protein, 24 aa, synthetic construct
  REGION 1..24: note = cell penetrating peptide (CPP)
LGTYTQDFNKFHTFPQTAIGVGAP

SEQ ID NO: 18 - protein, 12 aa, synthetic construct
  REGION 1..12: note = cell penetrating peptide (CPP)
TSPLNIHNGQKL

SEQ ID NO: 19 - protein, 12 aa, synthetic construct
  REGION 1..12: note = cell penetrating peptide (CPP)
NSAAFEDLRVLS

SEQ ID NO: 20 - protein, 30 aa, synthetic construct
  REGION 1..30: note = cell penetrating peptide (CPP)
WEAKLAKALAKALAKHLAKALAKALKACEA

SEQ ID NO: 21 - protein, 16 aa, synthetic construct
  REGION 1..16: note = cell penetrating peptide (CPP)
AAVALLPAVLLALLAP

SEQ ID NO: 22 - protein, 10 aa, synthetic construct
  REGION 1..10: note = cell penetrating peptide (CPP)
VPMLKPMLKE

SEQ ID NO: 23 - protein, 18 aa, synthetic construct
  REGION 1..18: note = cell penetrating peptide (CPP)
KLALKLALKALKAALKLA

SEQ ID NO: 24 - protein, 27 aa, synthetic construct
  REGION 1..27: note = cell penetrating peptide (CPP)
GALFLGFLGAAGSTMGAWSQPKKKRKV

SEQ ID NO: 25 - protein, 16 aa, synthetic construct
  REGION 1..16: note = cell penetrating peptide (CPP)
AAVALLPAVLLALLAP

SEQ ID NO: 26 - protein, 9 aa, synthetic construct
  REGION 1..9: note = cell penetrating peptide (CPP)
VQRKRQKLM

SEQ ID NO: 27 - protein, 21 aa, synthetic construct
  REGION 1..21: note = cell penetrating peptide (CPP)
KETWWETWWTEWSQPKKKRKV

SEQ ID NO: 28 - protein, 15 aa, synthetic construct
  REGION 1..15: note = cell penetrating peptide (CPP)
SDLWEMMMVSLACQY

SEQ ID NO: 29 - protein, 16 aa, synthetic construct
  REGION 1..16: note = cell penetrating peptide (CPP)
RQIKIWFQNRRMKWKK

SEQ ID NO: 30 - protein, 7 aa, synthetic construct
  REGION 1..7: note = cell penetrating peptide (CPP)
RRMKWKK

SEQ ID NO: 31 - protein, 7 aa, synthetic construct
  REGION 1..7: note = cell penetrating peptide (CPP)
RRRRRRR

SEQ ID NO: 32 - protein, 9 aa, synthetic construct
  REGION 1..9: note = cell penetrating peptide (CPP)
RRRRRRRRR

SEQ ID NO: 33 - protein, 16 aa, synthetic construct
  REGION 1..16: note = cell penetrating peptide (CPP)
RVIRVWFQNKRCKDKK

SEQ ID NO: 34 - protein, 28 aa, synthetic construct
  REGION 1..28: note = cell penetrating peptide (CPP)
MANLGYWLLALFVTMWTDVGLCKKRPKP

SEQ ID NO: 35 - protein, 18 aa, synthetic construct
  REGION 1..18: note = cell penetrating peptide (CPP)
LLIILRRRIRKQAHAHSK

SEQ ID NO: 36 - protein, 18 aa, synthetic construct
  REGION 1..18: note = cell penetrating peptide (CPP)
VRLPPPVRLPPPVRLPPP

SEQ ID NO: 37 - protein, 7 aa, synthetic construct
  REGION 1..7: note = cell penetrating peptide (CPP)
PKKKRKV

SEQ ID NO: 38 - protein, 18 aa, synthetic construct
  REGION 1..18: note = cell penetrating peptide (CPP)
RGGRLSYSRRRFSTSTGR

SEQ ID NO: 39 - protein, 10 aa, synthetic construct
  REGION 1..10: note = cell penetrating peptide (CPP)
RRLSYSRRRF

SEQ ID NO: 40 - protein, 17 aa, synthetic construct
  REGION 1..17: note = cell penetrating peptide (CPP)
AWSFRVSYRGISYRRSR

SEQ ID NO: 41 - protein, 14 aa, synthetic construct
  REGION 1..14: note = cell penetrating peptide (CPP)
YGRKKRRQRRRPPQ

SEQ ID NO: 42 - protein, 11 aa, synthetic construct
  REGION 1..11: note = cell penetrating peptide (CPP)
YGRKKRRQRRR

SEQ ID NO: 43 - protein, 9 aa, synthetic construct
  REGION 1..9: note = cell penetrating peptide (CPP)
RKKRRQRRR

SEQ ID NO: 44 - protein, 13 aa, synthetic construct
  REGION 1..13: note = cell penetrating peptide (CPP)
GRKKRRQRRRPPQ

SEQ ID NO: 45 - protein, 27 aa, synthetic construct
  REGION 1..27: note = cell penetrating peptide (CPP)
GWTLNSAGYLLGKINLKALAALAKKIL

SEQ ID NO: 46 - protein, 21 aa, synthetic construct
  REGION 1..21: note = cell penetrating peptide (CPP)
AGYLLGKINLKALAALAKKIL

SEQ ID NO: 47 - protein, 12 aa, synthetic construct
  REGION 1..12: note = cell penetrating peptide (CPP)
GWTLNSAGYLLG

SEQ ID NO: 48 - protein, 14 aa, synthetic construct
  REGION 1..14: note = cell penetrating peptide (CPP)
INLKALAALAKKIL

SEQ ID NO: 49 - protein, 34 aa, synthetic construct
  REGION 1..34: note = cell penetrating peptide (CPP)
DAATATRGRSAASRPTERPRAPARSASRPRRPVD

SEQ ID NO: 50 - protein, 26 aa, synthetic construct
  REGION 1..26: note = cell penetrating peptide (CPP)
DPKGDPKGVTVTVTVTVTGKGDPKPD

SEQ ID NO: 51 - protein, 8 aa, synthetic construct
  REGION 1..8: note = cell penetrating peptide (CPP)
KRGLKLRH

SEQ ID NO: 52 - protein, 9 aa, synthetic construct
  REGION 1..9: note = cell penetrating peptide (CPP)
RKKRRQRRR

SEQ ID NO: 53 - protein, 16 aa, synthetic construct
  REGION 1..16: note = cell penetrating peptide (CPP)
KRPAAIKKAGQAKKKK

SEQ ID NO: 54 - protein, 13 aa, synthetic construct
  REGION 1..13: note = cell penetrating peptide (CPP)
TRRQRTRRARRNR

SEQ ID NO: 55 - protein, 15 aa, synthetic construct
  REGION 1..15: note = cell penetrating peptide (CPP)
QTRRRERRAEKQAQW

SEQ ID NO: 56 - protein, 18 aa, synthetic construct
  REGION 1..18: note = cell penetrating peptide (CPP)
TAKTRYKARRAELIAERR

SEQ ID NO: 57 - protein, 22 aa, synthetic construct
  REGION 1..22: note = cell penetrating peptide (CPP)
MDAQTRRRERRAEKQAQWKAAN

SEQ ID NO: 58 - protein, 15 aa, synthetic construct
  REGION 1..15: note = cell penetrating peptide (CPP)
RRRRNRTRRNRRRVR

SEQ ID NO: 59 - protein, 19 aa, synthetic construct
  REGION 1..19: note = cell penetrating peptide (CPP)
KMTRAQRRAAARRNRWTAR

SEQ ID NO: 60 - protein, 12 aa, synthetic construct
  REGION 1..12: note = cell penetrating peptide (CPP)
RQARRNRRRRWR

SEQ ID NO: 61 - protein, 17 aa, synthetic construct
  REGION 1..17: note = cell penetrating peptide (CPP)
TRQARRNRRRRWRERQF

SEQ ID NO: 62 - protein, 37 aa, synthetic construct
  REGION 1..37: note = cell penetrating peptide (CPP)
ENARQGAARVTSWLGRQLRIAGKRLEGRSKTWFGAYA

SEQ ID NO: 63 - protein, 27 aa, synthetic construct
  REGION 1..27: note = cell penetrating peptide (CPP)
GALFLGWLGAAGSTMGAWSQPKKKRKV

SEQ ID NO: 64 - protein, 34 aa, synthetic construct; identical to SEQ ID NO: 49
  REGION 1..34: note = cell penetrating peptide (CPP)

SEQ ID NO: 65 - protein, 7 aa, synthetic construct
  REGION 1..7: note = cell penetrating peptide (CPP)
PKKKRKV

SEQ ID NO: 66 - protein, 16 aa, synthetic construct
  REGION 1..16: note = cell penetrating peptide (CPP)
RQIKIWFQNRRMKWKK

SEQ ID NO: 67 - protein, 10 aa, synthetic construct
  REGION 1..10: note = cell penetrating peptide (CPP)
NYRWRCKNQN

SEQ ID NO: 68 - protein, 22 aa, synthetic construct
  REGION 1..22: note = cell penetrating peptide (CPP)
SVKAQYKKNSDKHRLMRKRGLK

SEQ ID NO: 69 - protein, 24 aa, synthetic construct
  REGION 1..24: note = cell penetrating peptide (CPP)
LGTYTQDFNKFHTFPQTAIGVGAP

SEQ ID NO: 70 - protein, 18 aa, synthetic construct
  REGION 1..18: note = cell penetrating peptide (CPP)
LLIILRRRIRKQAHAHSK

SEQ ID NO: 71 - protein, 22 aa, synthetic construct
  REGION 1..22: note = cell penetrating peptide (CPP)
KCFQWQRNMRKVRGPPVSCIKR

SEQ ID NO: 72 - protein, 16 aa, synthetic construct
  REGION 1..16: note = cell penetrating peptide (CPP)
RHIKIWFQNRRMKWKK

SEQ ID NO: 73 - protein, 37 aa, synthetic construct
  REGION 1..37: note = cell penetrating peptide (CPP)
LLGDFFRKSKEKIGKEFKRIVQRIKDFLRNLVPRTES

SEQ ID NO: 74 - protein, 18 aa, synthetic construct
  REGION 1..18: note = cell penetrating peptide (CPP)
RGGRLSYSRRRFSTSTGR

SEQ ID NO: 75 - protein, 10 aa, synthetic construct
  REGION 1..10: note = cell penetrating peptide (CPP)
RRLSYSRRRF

SEQ ID NO: 76 - protein, 28 aa, synthetic construct
  REGION 1..28: note = cell penetrating peptide (CPP)
MVKSKIGSWILVLFVAMWSDVGLCKKRP

SEQ ID NO: 77 - protein, 42 aa, synthetic construct
  REGION 1..42: note = cell penetrating peptide (CPP)
YKQCHKKGGHCFPKEKICLPPSSDFGKMDCRWRWKCCKKGSG

SEQ ID NO: 78 - protein, 33 aa, synthetic construct
  REGION 1..33: note = cell penetrating peptide (CPP)
GDCLPHLKLCKENKDCCSKKCKRRGTNIEKRCR

SEQ ID NO: 79 - protein, 8 aa, synthetic construct
  REGION 1..8: note = cell penetrating peptide (CPP)
RKKRRQRR

SEQ ID NO: 80 - protein, 6 aa, synthetic construct
  REGION 1..6: note = cell penetrating peptide (CPP)
TRRQRT

SEQ ID NO: 81 - protein, 6 aa, synthetic construct
  REGION 1..6: note = cell penetrating peptide (CPP)
RRRERR

SEQ ID NO: 82 - protein, 9 aa, synthetic construct
  REGION 1..9: note = cell penetrating peptide (CPP)
KTRYKARRA

SEQ ID NO: 83 - protein, 8 aa, synthetic construct
  REGION 1..8: note = cell penetrating peptide (CPP)
TRRRERRA

SEQ ID NO: 84 - protein, 15 aa, synthetic construct
  REGION 1..15: note = cell penetrating peptide (CPP)
RRRRNRTRRNRRRVR

SEQ ID NO: 85 - protein, 6 aa, synthetic construct
  REGION 1..6: note = cell penetrating peptide (CPP)
ARRNRW

SEQ ID NO: 86 - protein, 12 aa, synthetic construct
  REGION 1..12: note = cell penetrating peptide (CPP)
RQARRNRRRRWR

SEQ ID NO: 87 - protein, 17 aa, synthetic construct
  REGION 1..17: note = cell penetrating peptide (CPP)
TRQARRNRRRRWRERQF

SEQ ID NO: 88 - protein, 10 aa, synthetic construct
  REGION 1..10: note = cell penetrating peptide (CPP)
WSQPKKKRKV

SEQ ID NO: 89 - protein, 6 aa, synthetic construct
  REGION 1..6: note = cell penetrating peptide (CPP)
SRPRRP

SEQ ID NO: 90 - protein, 7 aa, synthetic construct
  REGION 1..7: note = cell penetrating peptide (CPP)
PKKKRKV

SEQ ID NO: 91 - protein, 6 aa, synthetic construct
  REGION 1..6: note = cell penetrating peptide (CPP)
NRRMKW

SEQ ID NO: 92 - protein, 5 aa, synthetic construct
  REGION 1..5: note = cell penetrating peptide (CPP)
RWRCK

SEQ ID NO: 93 - skipped

SEQ ID NO: 94 - protein, 7 aa, synthetic construct
  REGION 1..7: note = cell penetrating peptide (CPP)
LRRRIRK

SEQ ID NO: 95 - protein, 6 aa, synthetic construct
  REGION 1..6: note = cell penetrating peptide (CPP)
MRKVRG

SEQ ID NO: 96 - protein, 8 aa, synthetic construct
  REGION 1..8: note = cell penetrating peptide (CPP)
NRRMKWKK

SEQ ID NO: 97 - protein, 8 aa, synthetic construct
  REGION 1..8: note = cell penetrating peptide (CPP)
FRKSKEKI

SEQ ID NO: 98 - protein, 5 aa, synthetic construct
  REGION 1..5: note = cell penetrating peptide (CPP)
RWRWK

SEQ ID NO: 99 - protein, 6 aa, synthetic construct
  REGION 1..6: note = cell penetrating peptide (CPP)
SKKCKR

SEQ ID NO: 100 - protein, 5 aa, synthetic construct
  REGION 1..5: note = cell penetrating peptide (CPP)
EKRCR

SEQ ID NO: 101 - protein, 8 aa, synthetic construct
  REGION 1..8: note = cell penetrating peptide (CPP)
CPKKKRKV

SEQ ID NO: 102 - protein, 7 aa, synthetic construct
  REGION 1..7: note = cell penetrating peptide (CPP)
CRRLRWR

SEQ ID NO: 103 - protein, 10 aa, synthetic construct
  REGION 1..10: note = cell penetrating peptide (CPP)
CRKKRRQRRR

SEQ ID NO: 104 - protein, 11 aa, synthetic construct
  REGION 1..11: note = cell penetrating peptide (CPP)
  BINDING 1: note = conjugated to Trt protecting group
  BINDING 7: note = comjugated to Acm protecting group
CRYRWRCKNQN

SEQ ID NO: 105 - protein, 20 aa, synthetic construct
  REGION 1..20: note = cell penetrating peptide (CPP)
CKVRVRVRVDPPTRVRERVK

SEQ ID NO: 106 - protein, 19 aa, synthetic construct
  REGION 1..19: note = cell penetrating peptide (CPP)
VKRGLKLRHVRPRVTRMDV

SEQ ID NO: 107 - protein, 9 aa, synthetic construct
  REGION 1..9: note = cell penetrating peptide (CPP)
RRKRRRRRK

SEQ ID NO: 108 - protein, 30 aa, synthetic construct
  REGION 1..30: note = cell penetrating peptide (CPP)
GAAEAAARVYDLGLRRLRQRRRLRRERVRA

SEQ ID NO: 109 - protein, 30 aa, synthetic construct
  REGION 1..30: note = cell penetrating peptide (CPP)
IREIMEKFGKQPVSLPARRLKLRGRKRRQR

SEQ ID NO: 110 - protein, 30 aa, synthetic construct
  REGION 1..30: note = cell penetrating peptide (CPP)
YLKVVRKHHRVIAGQFFGHHHTDSFRMLYD

SEQ ID NO: 111 - protein, 30 aa, synthetic construct
  REGION 1..30: note = cell penetrating peptide (CPP)
SKVRFCSGRKRPVRRRPEPQLKGIVTRLFS

SEQ ID NO: 112 - protein, 30 aa, synthetic construct
  REGION 1..30: note = cell penetrating peptide (CPP)
SMSVLEPGTAKKHKGGILRKGAKLFFRRRH

SEQ ID NO: 113 - protein, 30 aa, synthetic construct
  REGION 1..30: note = cell penetrating peptide (CPP)
QRKIGGRGRIISPYRTPVLRRHRYSIFRST

SEQ ID NO: 114 - protein, 30 aa, synthetic construct
  REGION 1..30: note = cell penetrating peptide (CPP)
QHVRIRVIKKKKVIMKKRKKLTLTRPTPLV

SEQ ID NO: 115 - protein, 30 aa, synthetic construct
  REGION 1..30: note = cell penetrating peptide (CPP)
FHFFPRRPRIHFRFPNRPFVPSRCNHRFPF

SEQ ID NO: 116 - protein, 39 aa, synthetic construct
  REGION 1..39: note = cell penetrating peptide (CPP)
FALLGDFFRKSKEKIGKEFKRIVQRIKDFLRNLVPRTES

SEQ ID NO: 117 - protein, 10 aa, synthetic construct
  REGION 1..10: note = cell penetrating peptide (CPP)
NYRWRCKNQN

SEQ ID NO: 118 - protein, 8 aa, synthetic construct
  REGION 1..8: note = cell penetrating peptide (CPP)
TRRRERRA

SEQ ID NO: 119 - protein, 8 aa, synthetic construct
  REGION 1..8: note = cell penetrating peptide (CPP)
NRRMKWKK

SEQ ID NO: 120 - protein, 8 aa, synthetic construct
  REGION 1..8: note = cell penetrating peptide (CPP)
FRKSKEKI

SEQ ID NO: 121 - protein, 20 aa, synthetic construct
  REGION 1..20: note = cell penetrating peptide (CPP)
CVKRGLKLRHVRPRVTRMDV